(19) United States
(12) Patent Application Publication
(10) Pub. No.: US 2021/0248656 A1
(43) Pub. Date: Aug. 12, 2021

(54) METHOD AND SYSTEM FOR AN INTERFACE FOR PERSONALIZATION OR RECOMMENDATION OF PRODUCTS

(71) Applicant: LULULEMON ATHLETICA CANADA INC., Vancouver (CA)

(72) Inventors: John MAKOWSKY, Vancouver (CA); Joseph John SANTRY, Vancouver (CA); Thomas McCarthy WALLER, Vancouver (CA); Sian Victoria ALLEN, Vancouver (CA); Erica Margaret BUCKERIDGE, Vancouver (CA); Siân Elizabeth GORDON, Vancouver (CA); Todd James SMITH, Vancouver (CA); Peder Richard Douglas SANDE, Vancouver (CA); Amanda Susanne CASGAR, Vancouver (CA); Robert John GATHERCOLE, Vancouver (CA)

(21) Appl. No.: 17/191,515

(22) Filed: Mar. 3, 2021

Related U.S. Application Data
(63) Continuation-in-part of application No. PCT/CA2020/051454, filed on Oct. 29, 2020.
(60) Provisional application No. 63/052,836, filed on Jul. 16, 2020.
(60) Provisional application No. 62/928,210, filed on Oct. 30, 2019.
(60) Provisional application No. 63/052,836, filed on Jul. 16, 2020.

(51) Int. Cl. G06Q 30/06; G06Q 10/10; G06Q 30/02; G06Q 10/08; G06N 20/00; G06F 30/12; G06F 3/0482; H04N 21/234

(52) U.S. Cl. CPC G06Q 30/0621; G06Q 10/101; G06Q 30/0201; G06Q 10/0875; G06Q 30/0643; G06Q 50/04; G06N 20/00; G06F 30/12; G06F 3/0482; H04N 21/234; G06Q 30/0631

(57) ABSTRACT

There is described a system for providing an interface with personalized products or product recommendations by capturing user data with sensors and computing a physical and/or emotional signature of the user. The user data comprises at least image data, text input, biometric data, and audio data, and may have been captured using one or more sensors on the user device. The user data is processed using at least: facial analysis; body analysis; eye tracking; behavioural analysis; social network analysis; location analysis; user's activities analysis; speech analysis, and text analysis. Based on the user data, one or more states of one or more cognitive-affective competencies of the user may be determined. An is determined, based on the one or more states of the one or more cognitive-affective competencies of the user. Based on the physical and/or emotional signature, one or more personalized products or product recommendations are generating for improving the emotional signature or physical signature.

### CROSS-REFERENCE TO RELATED APPLICATIONS

This application is a continuation-in-part application of International (PCT) Patent Application No. PCT/CA2020/051454 entitled METHOD AND SYSTEM FOR AN INTERFACE TO PROVIDE ACTIVITY RECOMMENDATIONS filed Oct. 29, 2020 which claims the benefit of and priority to U.S. Provisional Application No. 63/052,836 filed Jul. 16, 2020 and U.S. Provisional Application No. 62/928,210 filed Oct. 30, 2019, the entire contents of which are hereby incorporated by reference.

### FIELD

The present disclosure relates generally to the field of computing, and in particular, to methods and systems for an interface for products and/or services that involves capturing user attributes and classifying the user attributes for personalization or recommendation of products and/or services. The methods and systems can involve an interface for capturing user measurements and activity with sensors and other data sources, determining a physical and emotional signature of a user for the personalization or recommendation of products and/or services, and generating the personalization or recommendation of products and/or services based on the physical and emotional signature of the user.

### BACKGROUND

Embodiments described herein relate to automated systems for personalization or recommendation of products and/or services that can involve detecting a person's physical characteristics and/or personality type, mood, and other emotional characteristics through the use of different information capture technology, including invasive and non-invasive sensors. As such, it is possible to attempt to establish a person's current physical and emotional state based on, for example, data for their heartrate, facial expressions or the tone of their voice as captured by various different sensors. A person who exhibits a state of physical and/or emotional wellbeing or desires to exhibit a state of physical and/or emotional wellbeing may be in need of product or service assistance, and there may exist different types of products, activities, coaching sessions, and therapies that may be used to assist the person in boosting their general physical and/or emotional fitness or wellbeing. In an aspect, embodiments described herein involve automated systems for providing personalization or recommendations for products and/or services with assistance tailored to an individual's specific personality and current state of physical and emotional wellbeing as captured by different information capture devices, such as sensors.

### SUMMARY

Embodiments relate to methods and systems with non-transitory memory storing data records for product personalization using a physical signature and emotional signature of a user.

Embodiments relate to methods and systems with non-transitory memory storing data records for product recommendation using a physical signature and emotional signature of a user.

In an aspect, there is provided a system for providing an interface for product personalization using a physical signature and emotional signature of a user. The system involves non-transitory memory storing an attributable database of at least one of product measurement records, movement features, perceptual preference features, physical signature features, emotional signature features, user records, product records, and generative design models. The system has a hardware processor programmed with executable instructions for an interface to obtain user data for a user session over a time period, transmit a product request for the user session, display a visualization of a product generated for the user session in response to the product request, and receive quantitative and qualitative feedback data on the product. The system has a hardware server coupled to the memory to access the attributable database. The hardware server is programmed with executable instructions to: in response to receiving the product request from the interface, select product category and product variables; extract user attributes from the user data for the user session and associate with the product variables, the user attributes comprising at least one of measurement metrics, movement metrics, perceptual preference metrics, physical signature metrics, emotional signature metrics, purchase history, and activity intent; compute target parameters for a target feel state for the user using the extracted user attributes; generate the product and associated manufacturing instructions by processing the extracted user attributes and the target parameters for the target feel state using the generative design models and the attributable database; transmit the visualization of the product to the interface; and update the attributable database or user records based on the feedback data on the product. The system involves a user device comprising one or more sensors for capturing the user data for the user session during the time period, and a transmitter for transmitting the captured user data to the interface of the hardware processor or the hardware server over the network to generate the product for the user session.

In some embodiments, the interface receives purchase instructions for the product, and wherein the hardware server transmits manufacturing instructions for the product in response to receiving purchase instructions.

In some embodiments, the hardware server generates the product and associated manufacturing instructions by generating bill of material files.

In some embodiments, the product comprises video content, and wherein the hardware server generates the product and associated code files by assembling content files for the video content.

In some embodiments, the interface receives a modification request for the product and wherein the hardware server updates the product and the associated manufacturing instructions based on the modification request.

In some embodiments, the user device captures the user data from a plurality of channels, wherein the user data comprises at least one of image data relating to the user, text input relating to the user, data defining physical or behavioural characteristics of the user, and audio data relating to the user.

In some embodiments, the hardware server is programmed with executable instructions to compute activity metrics, cognitive-affective competency metrics, and social metrics using the user data for the user session and the user attributes by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis; compute one or more states of one or more cognitive-affective competencies of the user based on the cognitive-affective competency metrics and the social metrics; compute the emotional signature metrics of the user based on the one or more states of the one or more cognitive-affective competencies of the user; and generating the product based on at least one of the emotional signature of the user, activity metrics, product records, and/or the user records.

In some embodiments, the hardware server generates the measurement metrics using at least one of 3D scanning, machine learning prediction, and user measuring.

In some embodiments, the product comprises a garment, and wherein the hardware server generates the measurement metrics using garment measuring to capture garment data for the product data.

In some embodiments, the hardware server generates the movement metrics based on at least one of inertial measurement unit (IMU) data, computer vision, pressure data, radio-frequency data.

In some embodiments, the hardware server extracts the user attributes from user data comprising at least one of purchase history, activity intent, interaction history, and review data for the user.

In some embodiments, the hardware server generates the perceptual preference metrics based on at least one of garment sensation, preferred hand feel, thermal preference, and movement sensation.

In some embodiments, the hardware server generates the emotional signature metrics based on at least one of personality data, mood state data, emotional fitness data, personal values data, goals data, and physiological data.

In some embodiments, the hardware processor computes a preferred sensory state as part of the extracted user attributes.

In some embodiments, the hardware server computes social signature metrics, connectedness metrics, and/or resonance signature metrics.

In some embodiments, the user device connects to or integrates with an immersive hardware device that captures audio data, the image data and data defining physical or behavioural characteristics of the user as part of the user data.

In some embodiments, the non-transitory memory has a content repository and the hardware server has a content curation engine that generates content as part of the product and transmits the generated content to the interface.

In some embodiments, the hardware processor receives object identification data and computes a preferred sensory state as part of the object identification data.

In some embodiments, the product comprises content for display or playback on the hardware processor or the user device.

In some embodiments, the product relates to a garment, wherein the attributable database comprises simulated garment records, wherein the hardware server generates simulated product options as part of the product and associated manufacturing instructions, and wherein the interface displays a visualization of the simulated product options.

In some embodiments, the simulated product options comprise at least one of soft body physics simulation, hard body physics simulation, static 3D viewer and AR/VR experience content.

In some embodiments, the hardware server extracts product variables or attributes using multimodal feature extraction and classifies the product variables and attributes.

In some embodiments, the hardware server classifies different types of data streams for the user data for multimodal feature extraction.

In some embodiments, the user data comprises image data relating to the user, text input relating to the user, data defining physical or behavioural characteristics of the user, and audio data relating to the user. The hardware server extracts the user attributes using multimodal feature extraction that: for the image data and the data defining the physical or behavioural characteristics of the user, implements at least one of: facial analysis; body analysis; eye tracking; behavioural analysis; social network or graph analysis; location analysis; user activity analysis; for the audio data, implements voice analysis; and for the text input implements text analysis; and computes one or more states of one or more cognitive-affective competencies of the user based on the cognitive-affective competency metrics and the social metrics.

In some embodiments, the hardware server extracts the user attributes by computing the emotion signature metrics based on one or more states of one or more cognitive-affective competencies of the user and social metrics of the user.

In some embodiments, the non-transitory memory stores classifiers for generating data defining physical or behavioural characteristics of the user, and the hardware server extracts the user attributes by computing activity metrics, cognitive-affective competency metrics, and social metrics using the classifiers.

In some embodiments, the non-transitory memory stores a user model corresponding to the user and the hardware server computes the emotional signature metrics of the user using the user model.

In some embodiments, the system has one or more modulators in communication with one or more ambient fixtures to change the external sensory environment based on the product, the one or more modulators being in communication with the hardware server to automatically modulate the external sensory environment of the user during the user session.

In some embodiments, the one or more ambient fixtures comprise at least one of a lightening fixture, an audio system, an aroma diffuser, a temperature regulating system.

In some embodiments, the system has a plurality of data channels, the data channels for a plurality of different types of sensors for capturing different types of user data during the user session, each of the plurality of devices transmitting the captured different types of user data to the hardware server over the network to generate the product.

In some embodiments, the hardware server is configured to determine an emotional signature of one or more additional users; determine users with similar emotional signatures; predict connectedness between users with similar emotional signatures; and generate the product using data corresponding to the users with similar emotional signatures.

In some embodiments, the interface can transmit another product request for the user session, and provide an additional visualization for another product for the user session received in response to the other product request.

In some embodiments, the product comprises a program for display or playback on a computing device, wherein the program comprises two or more phases, each phase having a different content, intensity, or duration.

In some embodiments, user data comprises personality type data, wherein the hardware server computes the emotional signature metrics by determining, based on the user data, a personality type of the user by comparing the personality type data to stored personality type data indicative of correlations between personality types and personality type data.

In some embodiments, the hardware server computes as part of the emotional signature metrics at least one of: one or more mood states of the user, one or more attentional states of the user, one or more prosociality states of the user, one or more motivational states of the user, one or more reappraisal states of the user, and one or more insight states of the user.

In some embodiments, the interface is a coaching application for improving the wellbeing of the user based on the product and at least one of the physical signature metrics, the emotional signature metrics, and perceptual preference metrics.

In an aspect, there is provided a method for providing an interface for generating a product. The method involves storing an attributable database of product measurement records, movement features, perceptual preference features, physical and emotional signature features, user records, and generative design models in memory; capturing user data for a user session over a time period; in response to receiving a product request from an interface, select product category and product variables; extracting user attributes from the user data and associate with the product variables, the user attributes comprising at least one of measurement metrics, movement metrics, perceptual preference metrics, and physical and emotional signature metrics; computing target parameters for a target feel state for the user using the extracted user attributes; generating a product and associated manufacturing instructions by processing the extracted user attributes and the target parameters for the target feel state using the generative design models and the attributable database, wherein generating the product is based on an emotional signature and physical signature of the user; displaying a visualization of the product at the interface with a selectable purchase option; receiving purchase instructions for the product in response to selection of the selectable purchase option at the interface; transmitting manufacturing instructions for the product to a manufacturing queue to trigger production and delivery of the product; receiving feedback data on the product; and updating the attributable database or user model based on the feedback data.

In some embodiments, the method involves receiving a modification request for the product at the interface; and updating the product and the associated manufacturing instructions based on the modification request.

In an aspect, there is provided a system for providing an interface with product recommendations. The system involves non-transitory memory storing an attributable database of at least one of product measurement records, movement features, perceptual preference features, physical and emotional signature features, user records, and generative design models. The system involves a hardware processor programmed with executable instructions for an interface to obtain user data for a user session over a time period, transmit a product request for the user session, provide product recommendations for the user session in response to the product request, receive a selected product of the product recommendations; and receive feedback data on the selected product. The system involves a hardware server coupled to the memory to access the attributable database. The hardware server is programmed with executable instructions to: in response to receiving the product request from the interface, select product category and product variables; extract user attributes from the user data for the user session and associate with the product variables, the user attributes comprising at least one of measurement metrics, movement metrics, perceptual preference metrics, and physical and emotional signature metrics; compute target parameters for a target feel state for the user using the extracted user attributes; compute product recommendations using a recommendation system to process the extracted user attributes and the target parameters for the target feel state, the product recommendations computed using an emotional signature and a physical signature; transmit the product recommendations to the interface over a network; receive notification of the selected product from the interface; receive feedback data on the selected product; and update the attributable database based on the feedback data on the selected product. The system involves at least one data channels with one or more sensors for capturing user data during the time period, and a transmitter for transmitting the captured user data to the interface of the hardware processor or the hardware server over the network to compute the product recommendations.

In some embodiments, the user attributes comprise quantitative user attributes of physical metrics and qualitative user attributes, the qualitative user attributes comprising the emotional signature metrics and/or perceptual preferences of the user.

In some embodiments, the interface further comprises a voice interface for communicating the product recommendations and the product request.

In some embodiments, the hardware server generates the selected product by processing the extracted user attributes and the target parameters for the target feel state using generative design models and the attributable database.

In some embodiments, the hardware server receives personalization data to generate the selected product.

In some embodiments, the hardware server generates the selected product and associated code files by generating bill of material files.

In some embodiments, the hardware server generates the selected product and associated code files by assembling content files.

In some embodiments, the interface receives a modification request for the selected product and wherein the hardware server updates the selected product and the associated code files based on the modification request.

In some embodiments, the hardware server is programmed with executable instructions to compute activity metrics, cognitive-affective competency metrics, and social metrics using the user data for the user session and the user attributes by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis; compute one or more states of one or more cognitive-affective competencies of the user based on the cognitive-affective competency metrics and the social metrics; compute the emotional signature metrics of the user based on the one or more states of the one or more cognitive-affective competencies of the user; and computing the product recommendations based on the emotional signature of the user, the activity metrics, the product records, and the user records.

In some embodiments, the hardware server generates the measurement metrics using at least one of 3D scanning, machine learning prediction, user measuring, and garment measuring.

In some embodiments, the hardware server generates the movement metrics based inertial measurement unit (IMU) data, computer vision, pressure data, radio-frequency data.

In some embodiments, the hardware server extracts the user attributes from user data comprising at least one of purchase history, activity intent, interaction history, and review data for the user.

In some embodiments, the hardware server generates the perceptual preference metrics based on at least one of movement, touch, temperature, sight, smell, sound, taste garment sensation, preferred handfeel, thermal preference, and movement sensation.

In some embodiments, the hardware server generates the emotional signature metrics based on at least one of personality data, mood state data, emotional fitness data, personal values data, goals data, and physiological data.

In some embodiments, the hardware processor computes a preferred sensory state as part of the extracted user attributes.

In some embodiments, the emotional signature metrics comprise social signature metrics, connectedness metrics, and/or resonance signature metrics.

In some embodiments, the user device connects to or integrates with an immersive hardware device that captures audio data, the image data and data defining physical or behavioural characteristics of the user as part of the user data.

In some embodiments, the selected product comprises content for display or playback on a computing device.

In some embodiments, the non-transitory memory has a content repository and the hardware server has a content curation engine that generates content as part of the selected product and transmits the generated content to the interface.

In some embodiments, the hardware processor receives object identification data and computes a preferred sensory state as part of the user data.

In some embodiments, the attributable database comprises simulated garment records, wherein the hardware server generates simulated product options as part of the product recommendations, and wherein the interface displays a visualization of the simulated product options.

In some embodiments, the simulated product options comprise at least one of soft body physics simulation, hard body physics simulation, static 3D viewer and AR/VR experience content.

In some embodiments, the user data comprises image data relating to the user, text input relating to the user, data defining physical or behavioural characteristics of the user, and audio data relating to the user, and wherein the hardware server extracts the user attributes using multimodal feature extraction that: for the image data and the data defining the physical or behavioural characteristics of the user, implements at least one of: facial analysis; body analysis; eye tracking; behavioural analysis; social network or graph analysis; location analysis; user activity analysis; for the audio data, implements voice analysis; and for the text input implements text analysis; and computing one or more states of one or more cognitive-affective competencies of the user based on the cognitive-affective competency metrics and the social metrics.

In some embodiments, the hardware server extracts the user attributes by computing the emotion signature metrics based on one or more states of one or more cognitive-affective competencies of the user and social metrics of the user.

In some embodiments, the non-transitory memory stores classifiers for generating data defining physical or behavioural characteristics of the user, and the hardware server extracts the user attributes by computing activity metrics, cognitive-affective competency metrics, and social metrics using the classifiers.

In some embodiments, the non-transitory memory stores a user model corresponding to the user and the hardware server computes the emotional signature metrics of the user using the user model.

In some embodiments, the system comprises a plurality of user devices, each having different types of sensors for capturing different types of user data during the user session, each of the plurality of devices transmitting the captured different types of user data to the hardware server over the network to generate the product recommendations.

In some embodiments, the hardware server is configured to determine an emotional signature and physical signature of one or more additional users; determine users with similar emotional signatures or physical signatures; predict connectedness between users with similar emotional signatures or physical signatures; and generate the product recommendations using data corresponding to the users with similar emotional signatures or physical signatures.

In some embodiments, the interface can transmit another product product request for the user session, and provide an additional visualization for other product recommendations for the user session received in response to the other product request.

In some embodiments, the product comprises a program for display or playback on the hardware processor or the user device, wherein the program comprises two or more phases, each phase having a different content, intensity, or duration.

In some embodiments, the user data comprises personality type data, wherein the hardware server computes the emotional signature metrics by determining, based on the user data, a personality type of the user by comparing the personality type data to stored personality type data indicative of correlations between personality types and personality type data.

In some embodiments, the hardware server computes as part of the emotional signature metrics at least one of: one or more mood states of the user, one or more attentional states of the user, one or more prosociality states of the user, one or more motivational states of the user, one or more reappraisal states of the user, and one or more insight states of the user.

In some embodiments, the interface is a coaching application for improving the wellbeing of the user based on the product recommendations and the emotional signature metrics.

This summary does not necessarily describe the entire scope of all aspects. Other aspects, features and advantages will be apparent to those of ordinary skill in the art upon review of the following description of specific embodiments.

### BRIEF DESCRIPTION OF THE DRAWINGS

Embodiments of the disclosure will now be described in conjunction with the accompanying drawings of which:

FIG. 1 shows a system for generating products or product recommendations for users based on their physical and emotional signatures, according to embodiments of the disclosure;

FIG. 2 shows a user device that may be used by users of the system of FIG. 1, according to embodiments of the disclosure;

FIG. 3 shows example emotional signature data between user data, cognitive-affective state detection types, cognitive-affective competencies, and personality type, according to embodiments of the disclosure;

FIG. 4 shows an example process for generating products according to embodiments of the disclosure;

FIG. 5A shows a flow diagram of a process for generating products for users based on their physical and emotional signatures, according to embodiments of the disclosure;

FIG. 5B shows a flow diagram of a process for generating products for users based on their physical and emotional signatures, according to embodiments of the disclosure;

FIG. 6 shows a flow diagram of a process for generating product recommendations for users based on their physical and emotional signatures, according to embodiments of the disclosure;

FIG. 7 shows a flow diagram of a process for generating product recommendations for users based on their physical and emotional signatures, according to embodiments of the disclosure;

FIG. 8 shows information capture processes for generating products or recommendations for users based on their physical and emotional signatures, according to embodiments of the disclosure;

FIG. 9 shows processes for manufacturing products for users according to embodiments of the disclosure;

FIG. 10 shows processes of content delivery for users according to embodiments of the disclosure;

FIG. 11 shows processes for capturing feedback data according to embodiments of the disclosure;

FIG. 12 shows a diagram of an example computing device;

FIG. 13 shows a system for generating products or product recommendations for users based on their physical and emotional signatures, according to embodiments of the disclosure;

FIG. 14 shows an example interface that provides product recommendations according to embodiments of the disclosure; and

FIG. 15 shows an example interface that provides personalized products according to embodiments of the disclosure.

### DETAILED DESCRIPTION

Embodiments relate to methods and systems for product personalization or recommendation for users based on physical and emotional signatures computed using user data captured by sensors or other means, such as for example text data input received from an interactive questionnaire at an interface. While various embodiments of the disclosure are described below, the disclosure is not limited to these embodiments, and variations of these embodiments may well fall within the scope of the disclosure which is to be limited only by the appended claims. Embodiments described herein can be used for personalization or recommendations of products and/or services. The term product as used herein can refer to products and/or services. An example product is a garment, such as a shirt, pants, jacket, bra, underwear, hats, gloves, scarfs, and other wearable products, or other types of products, such as a yoga mat to challenge and support the stage of the user in their practice and built for their unique body type, activity preferences, physical characteristics such as height. Further examples include an eyewear, a bag, fitness equipment, meal kits, food, drinks, footwear, self-care products, nutritional supplements, and so on. An example service is video content, such as video content for exercise classes or activities. Further examples include e-learning or coaching services with emotional fitness content. The content can relate to an activity, learning, business, romantic partners, coaches or mentors based on a social signature recommending humans you may resonate with The service can relate to diet plans, training plans, and other self-care services, including spa treatments.

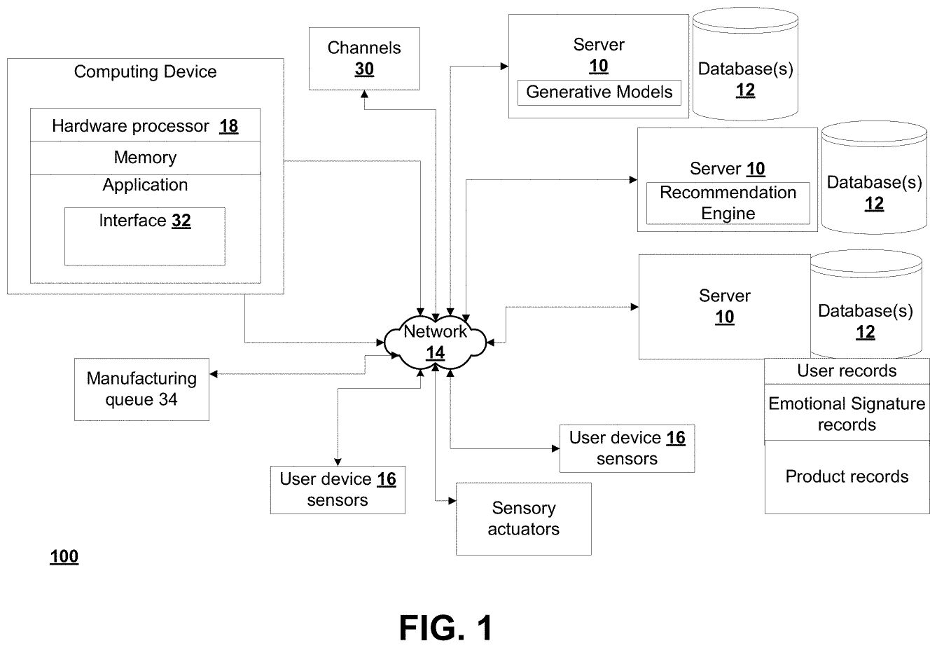

FIG. 1 shows an embodiment of a system 100 for product personalization or recommendation using physical and emotional data from users. The system 100 comprises hardware servers 10, databases 12 stored on non-transitory memory, a network 14, and user devices 16. The user device 16 can be an immersive hardware device to interact with users and capture user data. Servers 10 have hardware processors that are communicatively coupled to databases 12 stored on the non-transitory memory, and are operable to access data stored on databases 12. Servers 10 are further communicatively coupled to user devices 16 via network 14 (such as the Internet). Thus, data may be transferred between servers 10 and user devices 16 by transmitting the data using network 14. The servers 10 couples to one or more hardware processors 18 with an interface 32 and non-transitory computer readable storage medium storing instructions to configure the processor 18. The servers 100 couple to user devices 16 and one or more hardware processors 18 for collecting sensor data, and exchanging data and commands with other components of the system 100.

The hardware processor 18 has an interface 32 to provide visualizations of products or recommendations generated based on user data and physical and emotional signature metrics. The server 10 can access user data stored in the memory (as databases 12) to determine a physical and emotional signature of a user. The server 10 can generate the product or recommendations using the physical and emotional signature of a user. The server 10 can generate the product or recommendations by accessing a non-transitory memory storing a set of product records and the physical and emotional signature data of the user in databases 12. The interface 32 can display visual elements corresponding to a personalized product or recommendations generated based on the physical and emotional signature of the user, or otherwise communicate product recommendations, such as via audio data or video data. The display of the visual elements at the interface 32 can be controlled by the hardware processor 18 based on products identified at server 10 by the physical and emotional signature of the user.

The physical and emotional signature of a user can be computed by server 10 using different components, dimensions, and sub-dimensions extracted from user data. Example components include physical parameters (e.g. size, volume, weight, height, acceleration data, heart rate), activity (high and low intensity), preferences (e.g. how one wants to feel), movement parameters (legs, arms, breasts, body), sensory parameters (touch, taste, smell, sound, light. These metrics provide mechanistic components which differ for each person. There can be validated measures based on standards defined by parameters and functions, such as peak breast acceleration for a bra product.

Embodiments described herein provide improved methods and systems for an interface 32 for personalization of products or recommendations of products for users by the server 10 classifying user attributes from captured user data, determining physical and emotional signatures, determining product variables, and target feel states for users. The product variables can map to different physical and emotional signature variables of a user. For example, the product records can contain an electronic link to different physical and emotional signatures. The physical and emotional signature variables can be computed by server 10 using different components, dimensions, and sub-dimensions extracted from the user data.

The server 10 can associate products variables (weight, stretch, colour, temp, time, environment) with user sensation/feel states. There can be a data model used to connect the variables, for example. There can be lookup tables or logic gates. The correlations can be created through experimental research and encoded into mappings by server 10. The mappings can indicate a variable found to associate with a feel state. For example, if the user wanted to feel ‘at one with nature’, the server 10 can select increased airflow at the neck and an increased conductivity material.

Generally, according to embodiments of the disclosure, there are described methods and systems for personalization of products or recommendations of products, in part based on determining the emotional signature of a user. The emotional signature may be a composite metric derived from the combination of a measure of a personality type of the user (e.g. a measure of, for example, the user's openness/intellect, conscientiousness, extraversion, agreeableness, and neuroticism/emotional stability) and levels or states of cognitive-affective processes or competencies (e.g. attention, emotion regulation, awareness, compassion, etc.).

Generally, according to embodiments of the disclosure, there are described methods and systems for personalization of products or recommendations of products, in part based on determining the physical signature of a user. The physical signature may be a composite metric derived from the combination of a measure of different components, dimensions, and sub-dimensions extracted from user data. Example components include physical parameters (e.g. size, volume, weight, height, acceleration data, heart rate), activity (high and low intensity), preferences (e.g. how one wants to feel), movement parameters (legs, arms, breasts, body), sensory parameters (touch, taste, smell, sound, light. These metrics provide mechanistic components which differ for each person. There can be standards defined by parameters and functions that map to different physical signature data points.

The server 10 can automate the personalization or recommendation of products based on determining the emotional signature and physical signature of a user using different types of data and capture methods.

For example, text data input can be received from an interactive questionnaire at the interface 32. There can be a number of questions on the components of the emotional signature and physical signature. Example components of the emotional signature can be Awareness, Response and Compassion, and an interactive questionnaire can be provided and answers are rated on Likert scales (1-5, 1-7), and scores on A, R, C are summed and normalized to a % out of 100. These components may be sub-divided into dimensions, which may be further sub-divided into sub-dimensions, with the scores for the individual sub-dimensions being used to calculate scores for their corresponding dimensions and components. For example, Regulation may be sub-divided into Self-Management and Self-Regulation, and Self-Management may, in turn, be sub-divided into Effectiveness and Drive. The table below shows an example of potential dimensions and sub-dimensions of Awareness, Response and Compassion components of the emotional signature.

| Component | Dimension | Sub-Dimension | Description |
|---|---|---|---|
| Awareness | Intention | Purposefulness | Setting intentions, visioning and connecting |
| | | | to life purpose |
| | Attention | Mindfulness | Being present & observing inner and outer |
| | | | worlds curiously |
| | | Reflectiveness | Labelling experiences and expressing them |
| | | | in words |
| Regulation | Self- | Effectiveness | Setting long-term and short-term goals |
| | Management | Drive | Pursuing personal growth for its intrinsic |
| | | | value |
| | Self- | Focus | Maintaining stable attention on a task |
| | Regulation | Contextual Regulation | Sitting with and reframing difficult emotions |
| | | Acceptance | Separating your sense of self from your |
| | | | emotions |
| Compassion | Compassion | Relatedness | Maintaining close, trusting relationships with |
| | for Others | | loved ones |
| | | Perceptiveness | Understanding and taking the perspective of |
| | | | others |
| | | Empatheticness | Feeling the emotions of others |
| | | Sympatheticness | Caring about the suffering of others |
| | Self- | Self-Compassionateness | Being kind, positive and non-judgmental |
| | Compassion | | with yourself |
| | | Wisdom | Understanding the role of suffering as a |
| | | | teacher |

Various means of turning these scores into metrics for the user include clustering into persona types based on data trends, presenting overall scores, and presenting dominant components based on raw scores (e.g. A=highest). Metrics may be based on scores for individual dimensions or sub-dimensions, for example metrics may distinguish users with equal scores for Awareness scores but varying scores for Intention and Attention.

In some implementations, metrics can be determined by behavioural tasks for auto-assessment of A, R and C (e.g. breathing exercise), passive assessment via measurement (HRV, face recognition etc.), and inner-circle assessment, whereby the questionnaire is filled out by 1-5 people close to you and scores are computed to reflect true awareness vs current self-awareness (like a 360 review).

The examples relate to different state competencies, using questionnaires adapted from research-validated scales. A long-form interactive questionnaire can be 61 questions or more, and the interactive questionnaire asks questions on the components of Awareness, Response and Compassion. Answers are rated on scales and scores on A, R, C and summed and normalized. There can be different operations for turning these scores into metrics for the user. Examples include clustering into persona types based on data trends, presenting overall scores, and presenting dominant components based on raw scores. A short-form interactive questionnaire asks 14 questions (as an example), and functions the same way, just with the number of questions cut down.

Other example interactive questionnaires and data capture operations include: behavioural tasks for auto-assessment of A, R and C (e.g. breathing exercise), passive assessment via measurement (HRV, face recognition), and inner-circle assessment, whereby the questionnaire is filled out by 1-5 people close to you and scores are computed to reflect true awareness as compared current self-awareness to provide a comprehensive assessment of the user.

Another example relates to different trait competencies. Trait data can be combined with state data, and operations for collecting trait data and computing trait metrics can be similar to state metrics, such as including these variables in the clustering process, or process of determining dominant attributes.

In order for the interface 32 to generate visualizations for personalization or recommendations of products, user devices 16 may use one or more sensors to capture user data relating to the user. The sensors may include, for example, audio sensors (such as a microphone), optical sensors (such as a camera), tactile sensors (such as a user interface), biometric sensors (such as a heart monitor, blood pressure monitor, skin wetness monitor, electroencephalogram (EEG) electrode, etc.), location/position sensors (such as GPS) and motion detection or motion capturing sensors (such as accelerometers) for obtaining the user data. The user data may then be transmitted to server 10 and processed (using, for example, any of various face and body modelling or analysis techniques) and compared to stored, reference user data to determine the user's physical characteristics, personality type and states of cognitive-affective competencies. For example, the processed user data may be used to determine one or more of the user's current mood states which in turn may assist in determining the user's personality type and states of cognitive-affective competencies, or for example it may determine a movement profile of the body which may in turn assist in determining product attributes for the creation of a personalized product. The server 10 can generate personalization or recommendations of products using the user's physical characteristics, personality type and states of cognitive-affective competencies.

In addition, by monitoring the individual's physical and emotional signatures over time, the methods and systems described herein may determine whether the physical and emotional signature is improving or deteriorating. The individual's “baseline” physical and emotional signature may be calculated over time, for example by collecting averages on the individual's states or levels of cognitive-affective competencies. Through repeated interventions over time, the levels of these competencies may increase. Thus, a user's baseline physical and emotional signature may improve over time. The baseline physical and emotional signature may comprise the levels or states of the user's cognitive-affective competencies, in combination with the user's personality type, averaged over a period of time, or may comprise of biomechanical profiles, in combination with users feel preferences, averaged over a period of time.

There can be different interventions that take place based on the diagnosis of physical or emotional state.

After determining the user's physical and emotional signatures, personalized products may be generated by the server 10 or recommendations of products may be generated by the server 10, including products for improving the physical or emotional signature or reaching a target feel state. For example, the personalized product or recommendations may be based on recommendations that have shown, in connection with similar physical or emotional signatures of other users, to show an improvement in the physical or emotional signature in response to the recommendations or products. Depending on the evolution of the user's physical or emotional signature over time, the recommendations may be adjusted. For example, a product that has proven to lead to an improvement in that user's emotional signature, may also be generated for a different user that is exhibiting a similar emotional signature.

A number of users of system 100 may use interfaces 32 and/or user devices 16 to exchange data and commands with servers 10 in manners described in further detail below. While two user devices 16 are shown in FIG. 1, the system 100 is adaptable to be used by any suitable number of user devices 16, and even a single user device 16. Furthermore, while system 100 shows two servers 10 and two databases 12, system 100 extends to any suitable number of servers 10 and databases 12 (such as a single server communicatively coupled to a single database). Furthermore, while system 100 shows an interface 32, system 100 extends to any suitable number of interfaces 22 to provide visualizations.

In some embodiments, the function of databases 12 may be incorporated with that of servers 10 with non-transitory storage devices or memory. In other words, servers 10 may store the user data located on databases 12 within internal memory and may additionally perform any of the processing of data described herein. However, in the embodiment of FIG. 1, servers 10 are configured to remotely access the contents of databases 12 when required.

The server 10 can receive purchase instructions from the interface 32 at processor 18, and can transmit manufacturing instructions for the product to a manufacturing queue 34 in response to receiving purchase instructions.

Accordingly, the server 10 can provide product personalization using a physical signature and emotional signature of a user. Further, the serer 10 can provide product recommendation using a physical signature and emotional signature of a user.

The server 10 connects with the interface 32 for product personalization using a physical signature and emotional signature of a user. The server 10 connects non-transitory memory storing an attributable database 12 of at least one of product measurement records, movement features, perceptual preference features, physical signature features, emotional signature features, user records, product records, and generative design models.

The processor 18 is programmed with executable instructions for the interface 32 to obtain user data for a user session over a time period, and transmit a product request for the user session. The interface 32 displays a visualization of a product generated for the user session in response to the product request, and receives quantitative and qualitative feedback data on the product. The server 10 is coupled to the memory to access the attributable database 12. The hardware server 10 is programmed with executable instructions to, in response to receiving the product request from the interface, select product category and product variables. The server 10 can extract user attributes from the user data for the user session and associate with the product variables. The user attributes can be measurement metrics, movement metrics, perceptual preference metrics, physical signature metrics, emotional signature metrics, purchase history, and activity intent. The server 10 computes target parameters for a target feel state for the user using the extracted user attributes. The server 10 generates the product and associated manufacturing instructions by processing the extracted user attributes and the target parameters for the target feel state using the generative design models and the attributable database. The server 10 transmits the visualization of the product to the interface, and updates the attributable database or user records based on the feedback data on the product. The server 10 connects to a user device 16 with one or more sensors for capturing the user data for the user session during the time period. The user device 16 has a transmitter for transmitting the captured user data to the interface 32 of the hardware processor 18 or the hardware server 10 over the network to generate the product for the user session.

In some embodiments, the interface 32 receives purchase instructions for the product, and the hardware server 10 transmits manufacturing instructions for the product in response to receiving purchase instructions. In some embodiments, the hardware server 10 generates the product and associated manufacturing instructions by generating bill of material files. In some embodiments, the interface 32 receives a modification request for the product and the hardware server 10 updates the product and the associated manufacturing instructions based on the modification request.

In some embodiments, the product comprises video content, and the hardware server 10 generates the product and associated code files by assembling content files for the video content.

In some embodiments, the user device 16 captures the user data from a plurality of channels 30. The user data can be image data relating to the user, text input relating to the user, data defining physical or behavioural characteristics of the user, and audio data relating to the user.

In some embodiments, the hardware server 10 is programmed with executable instructions to compute activity metrics, cognitive-affective competency metrics, and social metrics using the user data for the user session and the user attributes. The server 10 can do this computation by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis. The server 10 can compute one or more states of one or more cognitive-affective competencies of the user based on the cognitive-affective competency metrics and the social metrics. The server 10 can compute the emotional signature metrics of the user based on the one or more states of the one or more cognitive-affective competencies of the user. The server 10 generates the product based on at least one of the emotional signature of the user, activity metrics, product records, and/or the user records.

In some embodiments, the hardware server 10 generates the measurement metrics using at least one of 3D scanning, machine learning prediction, and user measuring.

In some embodiments, the product comprises a garment, and the hardware server 10 generates the measurement metrics using garment measuring to capture garment data for the product data.

In some embodiments, the hardware server 10 generates the movement metrics based on at least one of inertial measurement unit (IMU) data, computer vision, pressure data, radio-frequency data.

In some embodiments, the hardware server 10 extracts the user attributes from user data such as purchase history, activity intent, interaction history, and review data for the user.

In some embodiments, the hardware server 10 generates the perceptual preference metrics based on at least one of garment sensation, preferred hand feel, thermal preference, and movement sensation. In some embodiments, the hardware server 10 generates the emotional signature metrics based on at least one of personality data, mood state data, emotional fitness data, personal values data, goals data, and physiological data. In some embodiments, the hardware processor 10 computes a preferred sensory state as part of the extracted user attributes. In some embodiments, the hardware server 10 computes social signature metrics, connectedness metrics, and/or resonance signature metrics.

In some embodiments, the user device 16 connects to or integrates with an immersive hardware device that captures audio data, the image data and data defining physical or behavioural characteristics of the user as part of the user data.

In some embodiments, the database 12 has a content repository and the hardware server 10 has a content curation engine that generates content as part of the product and transmits the generated content to the interface 32. In some embodiments, the product comprises content for display or playback on the hardware processor 18 or the user device 16. In some embodiments, the product comprises a program for display or playback on a computing device, wherein the program comprises two or more phases, each phase having a different content, intensity, or duration.

In some embodiments, the hardware processor 18 receives object identification data and computes a preferred sensory state as part of the object identification data.

In some embodiments, the product relates to a garment, wherein the attributable database comprises simulated garment records. The hardware server 10 generates simulated product options as part of the product and associated manufacturing instructions, and the interface 32 displays a visualization of the simulated product options.

In some embodiments, the simulated product options comprise at least one of soft body physics simulation, hard body physics simulation, static 3D viewer and AR/VR experience content.

In some embodiments, the hardware server 10 extracts product variables or attributes using multimodal feature extraction and classifies the product variables and attributes. In some embodiments, the hardware server 10 classifies different types of data streams for the user data for multimodal feature extraction.

In some embodiments, the user data comprises image data relating to the user, text input relating to the user, data defining physical or behavioural characteristics of the user, and audio data relating to the user. The hardware server 10 extracts the user attributes using multimodal feature extraction that: for the image data and the data defining the physical or behavioural characteristics of the user, implements at least one of: facial analysis; body analysis; eye tracking; behavioural analysis; social network or graph analysis; location analysis; user activity analysis; for the audio data, implements voice analysis; and for the text input implements text analysis; and computes one or more states of one or more cognitive-affective competencies of the user based on the cognitive-affective competency metrics and the social metrics.

In some embodiments, the hardware server 10 extracts the user attributes by computing the emotion signature metrics based on one or more states of one or more cognitive-affective competencies of the user and social metrics of the user.

In some embodiments, the non-transitory memory stores classifiers for generating data defining physical or behavioural characteristics of the user, and the hardware server 10 extracts the user attributes by computing activity metrics, cognitive-affective competency metrics, and social metrics using the classifiers.

In some embodiments, the non-transitory memory stores a user model corresponding to the user and the hardware server 10 computes the emotional signature metrics of the user using the user model.

In some embodiments, the system 100 has one or more modulators in communication with one or more ambient fixtures to change the external sensory environment based on the product, the one or more modulators being in communication with the hardware server to automatically modulate the external sensory environment of the user during the user session. In some embodiments, the one or more ambient fixtures comprise at least one of a lightening fixture, an audio system, an aroma diffuser, a temperature regulating system.

In some embodiments, the system 100 has a plurality of data channels 30. The data channels corresponding to a plurality of different types of sensors for capturing different types of user data during the user session. Each of the plurality of data channels 30 transmitting the captured different types of user data to the hardware server 10 over the network to generate the product.

In some embodiments, the hardware server 10 is configured to determine an emotional signature of one or more additional users, determine users with similar emotional signatures, and predict connectedness between users with similar emotional signatures. The server 10 generates the product using data corresponding to the users with similar emotional signatures.

In some embodiments, the interface 32 can transmit another product request for the user session, and provide an additional visualization for another product for the user session received in response to the other product request.

In some embodiments, user data comprises personality type data, and the hardware server 10 computes the emotional signature metrics by determining, based on the user data, a personality type of the user by comparing the personality type data to stored personality type data indicative of correlations between personality types and personality type data.

In some embodiments, the hardware server 10 computes as part of the emotional signature metrics at least one of: one or more mood states of the user, one or more attentional states of the user, one or more prosociality states of the user, one or more motivational states of the user, one or more reappraisal states of the user, and one or more insight states of the user.

In some embodiments, the interface 32 is a coaching application for improving the wellbeing of the user based on the product and at least one of the physical signature metrics, the emotional signature metrics, and perceptual preference metrics.

In an aspect, the system 100 provides an interface 32 with product recommendations. The system 100 involves non-transitory memory storing an attributable database 12 of at least one of product measurement records, movement features, perceptual preference features, physical and emotional signature features, user records, and generative design models. The system 100 involves a hardware processor 18 programmed with executable instructions for an interface to obtain user data for a user session over a time period, transmit a product request for the user session, provide product recommendations for the user session in response to the product request, receive a selected product of the product recommendations; and receive feedback data on the selected product. The system 100 involves a hardware server 10 coupled to the memory to access the attributable database 12. The hardware server 10 is programmed with executable instructions to: in response to receiving the product request from the interface, select product category and product variables; extract user attributes from the user data for the user session and associate with the product variables, the user attributes comprising at least one of measurement metrics, movement metrics, perceptual preference metrics, and physical and emotional signature metrics; compute target parameters for a target feel state for the user using the extracted user attributes; compute product recommendations using a recommendation engine to process the extracted user attributes and the target parameters for the target feel state, the product recommendations computed using an emotional signature and a physical signature; transmit the product recommendations to the interface over a network; receive notification of the selected product from the interface; receive feedback data on the selected product; and update the attributable database 12 based on the feedback data on the selected product. The system 100 involves channels 30 with one or more sensors for capturing user data during the time period, and a transmitter for transmitting the captured user data to the interface 32 of the hardware processor 18 or the hardware server 10 over the network to compute the product recommendations.

In some embodiments, the user attributes comprise quantitative user attributes of physical metrics and qualitative user attributes, the qualitative user attributes comprising the emotional signature metrics and/or perceptual preferences of the user.

In some embodiments, the interface 32 further comprises a voice interface for communicating the product recommendations and the product request. In some embodiments, the hardware server 10 generates the selected product by processing the extracted user attributes and the target parameters for the target feel state using generative design models and the attributable database. In some embodiments, the hardware server 10 receives personalization data to generate the selected product. In some embodiments, the hardware server 10 generates the selected product and associated code files by generating bill of material files. In some embodiments, the hardware server 10 generates the selected product and associated code files by assembling content files.

In some embodiments, the interface 32 receives a modification request for the selected product and wherein the hardware server updates the selected product and the associated code files based on the modification request.

In some embodiments, the hardware server 10 is programmed with executable instructions to compute activity metrics, cognitive-affective competency metrics, and social metrics using the user data for the user session and the user attributes by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis; compute one or more states of one or more cognitive-affective competencies of the user based on the cognitive-affective competency metrics and the social metrics; compute the emotional signature metrics of the user based on the one or more states of the one or more cognitive-affective competencies of the user; and computing the product recommendations based on the emotional signature of the user, the activity metrics, the product records, and the user records.

In some embodiments, the hardware server 10 generates the measurement metrics using at least one of 3D scanning, machine learning prediction, user measuring, and garment measuring. In some embodiments, the hardware server generates 10 the movement metrics based inertial measurement unit (IMU) data, computer vision, pressure data, radio-frequency data. In some embodiments, the hardware server 10 extracts the user attributes from user data comprising at least one of purchase history, activity intent, interaction history, and review data for the user.

In some embodiments, the hardware server 10 generates the perceptual preference metrics based on at least one of movement, touch, temperature, sight, smell, sound, taste garment sensation, preferred handfeel, thermal preference, and movement sensation. In some embodiments, the hardware server 10 generates the emotional signature metrics based on at least one of personality data, mood state data, emotional fitness data, personal values data, goals data, and physiological data.

In some embodiments, the hardware processor 18 computes a preferred sensory state as part of the extracted user attributes. In some embodiments, the emotional signature metrics comprise social signature metrics, connectedness metrics, and/or resonance signature metrics.

In some embodiments, the user device 16 connects to or integrates with an immersive hardware device that captures audio data, the image data and data defining physical or behavioural characteristics of the user as part of the user data.

In some embodiments, the selected product comprises content for display or playback on a computing device. In some embodiments, the non-transitory memory has a content repository and the hardware server 10 has a content curation engine that generates content as part of the selected product and transmits the generated content to the interface 32. In some embodiments, the product comprises a program for display or playback on the hardware processor or the user device, wherein the program comprises two or more phases, each phase having a different content, intensity, or duration.

In some embodiments, the hardware processor 18 receives object identification data and computes a preferred sensory state as part of the user data.

In some embodiments, the attributable database comprises simulated garment records, wherein the hardware server 10 generates simulated product options as part of the product recommendations, and wherein the interface displays a visualization of the simulated product options. In some embodiments, the simulated product options comprise at least one of soft body physics simulation, hard body physics simulation, static 3D viewer and AR/VR experience content.

In some embodiments, the user data comprises image data relating to the user, text input relating to the user, data defining physical or behavioural characteristics of the user, and audio data relating to the user, and the hardware server 10 extracts the user attributes using multimodal feature extraction that: for the image data and the data defining the physical or behavioural characteristics of the user, implements at least one of: facial analysis; body analysis; eye tracking; behavioural analysis; social network or graph analysis; location analysis; user activity analysis; for the audio data, implements voice analysis; and for the text input implements text analysis; and computing one or more states of one or more cognitive-affective competencies of the user based on the cognitive-affective competency metrics and the social metrics.

In some embodiments, the hardware server 10 extracts the user attributes by computing the emotion signature metrics based on one or more states of one or more cognitive-affective competencies of the user and social metrics of the user.

In some embodiments, the non-transitory memory stores classifiers for generating data defining physical or behavioural characteristics of the user, and the hardware server 10 extracts the user attributes by computing activity metrics, cognitive-affective competency metrics, and social metrics using the classifiers.

In some embodiments, the non-transitory memory stores a user model corresponding to the user and the hardware server 10 computes the emotional signature metrics of the user using the user model.

In some embodiments, the system 100 comprises a plurality of user devices 16, each having different types of sensors for capturing different types of user data during the user session, each of the plurality of devices transmitting the captured different types of user data to the hardware server over the network to generate the product recommendations.

In some embodiments, the hardware server 10 is configured to determine an emotional signature and physical signature of one or more additional users; determine users with similar emotional signatures or physical signatures; predict connectedness between users with similar emotional signatures or physical signatures; and generate the product recommendations using data corresponding to the users with similar emotional signatures or physical signatures.

In some embodiments, the interface 32 can transmit another product product request for the user session, and provide an additional visualization for other product recommendations for the user session received in response to the other product request.

In some embodiments, the user data comprises personality type data, wherein the hardware server computes the emotional signature metrics by determining, based on the user data, a personality type of the user by comparing the personality type data to stored personality type data indicative of correlations between personality types and personality type data.

In some embodiments, the hardware server 10 computes as part of the emotional signature metrics at least one of: one or more mood states of the user, one or more attentional states of the user, one or more prosociality states of the user, one or more motivational states of the user, one or more reappraisal states of the user, and one or more insight states of the user.

In some embodiments, the interface 32 is a coaching application for improving the wellbeing of the user based on the product recommendations and the emotional signature metrics.

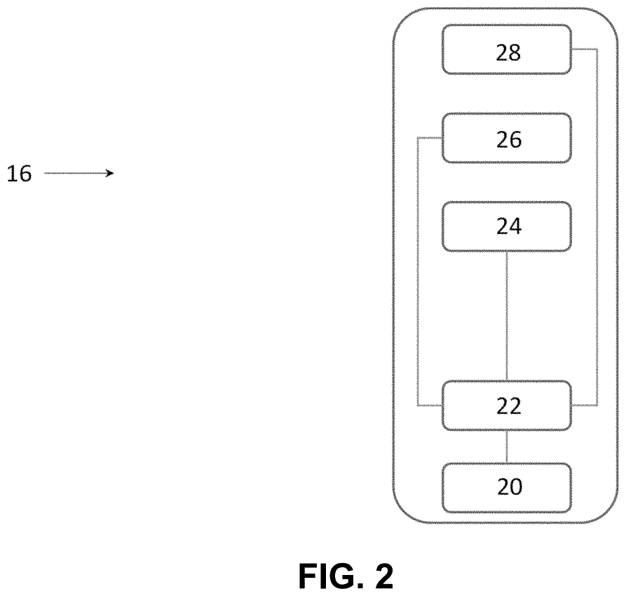

FIG. 2 shows an embodiment of a user device 16 in more detail. User device 16 includes a number of sensors, a hardware processor 22, and computer-readable medium 20 such as suitable computer memory storing computer program code. The user device 16 has a user interface 24 which may implement one or more functions or operations described in relation to interface 32 in some embodiments. The sensors 26, 28 include camera(s) 26, and microphone(s) 28, although the disclosure extends to other suitable sensors, such as biometric sensors (heart monitor, blood pressure monitor, skin wetness monitor etc.), any location/position sensors, motion detection or motion capturing sensors, and so on. The camera 26 can capture video and image data, for example. Processor 22 is communicative with each of sensors 26, 28 and is configured to control the operation of sensors 26, 28 in response to instructions read by processor 22 from non-transitory memory 20 and receive data from sensors 26, 28. According to some embodiments, user device 16 is a mobile device such a smartphone, although in other embodiments user device 16 may be any other suitable device that may be operated and interfaced with by a user. For example, user device 16 may comprise a laptop, a personal computer, a tablet device, a smart mirror, a smart display, a smart screen, a smart wearable, or an exercise device.

Sensors 26, 28 of user device 16 are configured to obtain user data relating to user. For example, microphone 28 may detect speech from user whereupon processor 22 may convert the detected speech into voice data. User may input text or other data into user device 16 via user interface 24, whereupon processor 22 may convert the user input into text data. Furthermore, camera 26 may capture images of user, for example when user is interfacing with user device 16. Camera 26 may convert the images into image data relating to user. The user interface 24 can send collected data from the different components of the user device 16 for transmission to the server 10 and storage in the database 12 as part of data records that are stored with an identifier for the user device 16 and/or user. The processor 22 can implement speech-to-text translation and run the analysis on the specific word usage/variance using natural language processing.

The system 100 captures user data for one or more users over a user session using one or more sensors 26, 28. In some embodiments, the system 100 provides an interface that displays a visualization of a product generated for the user session in response to the product request. In some embodiments, the system 100 provides an interface that displays a product recommendation generated for the user session in response to the product request. The interface can be user interface 24 of user device 16 in some embodiments, or an interface of a separate hardware device in some embodiments. The system 100 has non-transitory memory storing an attributable database of product measurement records, movement features, perceptual preference features, physical signature features, emotional signature features, product records, generative design models, physical signature records, emotional signature records, and user records storing user data received from a plurality of channels 30, at servers 10 and databases 12, for example.

The user data can involve a range of data captured during a time period of the user session (which can be combined with data from different user sessions and with data for different users). The user data can be image data relating to the user, text input relating to the user, data defining physical or behavioural characteristics of the user, and audio data relating to the user.

The system 100 has a hardware processor (which can be at user device 16) programmed with executable instructions for an interface (which can be user interface 24 for this example) for obtaining user data for a user session over a time period. The processor transmits a product request for the user session to the server 10, and updates its interface for providing visualizations products generated for the user sessions or product recommendations for the user session received in response to the product request.

The system 100 has a hardware server 10 coupled to the non-transitory memory (or database 12) to access the product records, the physical signature records, the emotional signature records, and the user records. The hardware server 10 is programmed with executable instructions to transmit the product recommendations to the interface 32 over a network 14 in response to receiving the product request from the interface. The hardware server 10 is programmed with executable instructions to compute the product recommendations by: computing activity metrics, physical metrics, cognitive-affective competency metrics, and social metrics using the user data for the user session and the user records. The hardware server 10 can extract user attributes from the user data to represent physical metrics of the user and cognitive metrics of the user. The hardware server 10 can use both physical metrics of the user and cognitive metrics of the user to determine the physical and emotional signatures for the user during the time period of the user session. The hardware server 10 can compute multiple physical and emotional signatures for the user at time intervals during the time period of the user session. The hardware server 10 compute multiple physical and emotional signatures which can trigger computation of updated products or recommendations and updated visualizations for the interface. The physical and emotional signatures each use both physical metrics of the user and cognitive metrics of the user during the time period of the user session to generate the products or recommendations.

The hardware server 10 can use user data captured during the user session and can also use user data captured during previous user sessions or user data for different users. The hardware server 10 can aggregate data from multiple channels to compute the products or recommendations to trigger updates to the interface 32 on the user device 16, or an interface on a separate hardware device in some examples.

The hardware server 10 can process different types of data by: for the image data and the data defining the physical or behavioural characteristics of the user, using at least one of: facial analysis; body analysis; eye tracking; behavioural analysis; social network or graph analysis; location analysis; user measurement analysis; for the audio data, using voice analysis; and for the text input using text analysis.

The hardware server 10 can compute one or more states of one or more physical characteristics of the user based on the physical metrics. The hardware server 10 can compute a physical signature of the user based on the one or more states of the one or more physical characteristics of the user and using the physical signature records. The physical signature records can store data for different components, dimensions, and sub-dimensions of physical characteristics of users.

The hardware server 10 can compute one or more states of one or more cognitive-affective competencies of the user based on the cognitive-affective competency metrics and the social metrics. The hardware server 10 can compute an emotional signature of the user based on the one or more states of the one or more cognitive-affective competencies of the user and using the emotional signature records.

The hardware server 10 can compute the product recommendations based on the physical and emotional signatures of the user, the measurement metrics, the product records, and the user records. The system has a user device comprising one or more sensors for capturing user data during the time period, and a transmitter for transmitting the captured user data to the interface or the hardware server over the network to compute the product recommendations.

In some embodiments, the system 100 has one or more modulators in communication with one or more ambient fixtures to change external sensory environment based on the generated product or recommendations, the one or more modulators being in communication with the hardware server 10 to automatically modulate the external sensory environment of the user. The changes to the sensory environment can be part of the product experience or integrated into a service. As noted, the term product as used herein can also include services. In some embodiments, the one or more ambient fixtures comprise at least one of a lightening fixture, an audio system, an aroma diffuser, a temperature regulating system.

The system 100 has multiple user devices 16 and each can have different types of sensors for capturing different types of user data during the user session. Each of the user devices 16 can be for transmitting the captured different types of user data to the hardware server 10 over the network 14 to compute the product or recommendations.

In some embodiments, the system 100 has multiple user devices 16 for a group of users. Each of the multiple user devices 16 has an interface for obtaining user data for a corresponding user of the group of users for the user session over the time period. The server 10 can provide product recommendations for the user session received in response to product requests from multiple user devices 16. The hardware server 10 transmits the product recommendations to the corresponding user interfaces 24 of the user devices 16 (or interface 32 of FIG. 1) in response to receiving the product request from the corresponding user interfaces 24. The server 10 can compute product recommendations for the group of users, and can suggest product recommendations for the group of users. The same product recommendations can be suggested for all the users of the group, or a set of users of the group with similar physical or emotional signatures as determined by the system 100 using similarity measurements.

In some embodiments, the hardware server 10 is configured to determine a physical or emotional signature of one or more additional users and determine users with similar physical or emotional signatures. The server 10 can predict connectedness between users with similar physical or emotional signatures and generate the product recommendations for the users with similar physical or emotional signatures.

In some embodiments, the interface 32 (or user interface 24 of the user device 16) can receive feedback on the product for the user session, transmit the feedback to the hardware server 10 to update generative models or recommendation engines. The feedback can be positive indicating approval of the product. The feedback can be negative indicating disapproval of the product. The server 10 can use the feedback for subsequent computations of product recommendations. The server 10 can store the feedback in the records of the database 12.

In some embodiments, the interface 32 (or user interface 24 of the user device 16) can transmit another product request for the user session, the server 10 can provide an additional product or recommendations for the user session in response to the other product request. The server 10 can transmit the additional product recommendations for the user session to update the interface 32.

In some embodiments, the interface 32 (or user interface 24 of the user device 16) obtains additional user data after providing the product recommendations for the user session, the additional user data captured during performance of the product recommendations by the user. The server 10 can use the additional user data captured after providing the product recommendations for the user session to re-compute the physical or emotional signature of the user for additional product recommendations.

In some embodiments, the interface 32 (or user interface 24 of the user device 16) transmits another product request for another user session, and provides an updated product or updated recommendations for the other user session received from the server 10 in response to the other product request. The updated product recommendations can be different than the initial product recommendations.

In some embodiments, the interface 32 is a coaching application and the one or more recommended products can be part of a virtual coaching program for the user to improve the user's wellbeing.

In some embodiments, the product recommendations relate to classes of products and can indicate classes selected from a set of classes stored in the product records. In some embodiments, a recommended product includes video content or a program with variety of content for the interface to guide user's interactions or experience for a prolong time. The content can be customized for the user. In some embodiments, a product recommendation includes a program with a variety of content to guide user's interactions or experience for a prolonged time. In some embodiments, the program comprises two or more phases, each phase having a different content, intensity, or duration. The phases can be customized content for the user.

In some embodiments, the server 10 can receive user data relating to one or more additional users from user devices 16 and determine, based on the processed user data, one or more states of one or more physical or cognitive-affective competencies of the one or more additional users. The server 10 can determine a physical and emotional signature of each of the one or more additional users and determine users with similar physical or emotional signatures. The server 10 can predict connectedness between users with similar physical or emotional signatures using similar models or measures stored in non-transitory memory. The server can generate one or more products or recommendations for transmission to interfaces of users with similar physical or emotional signatures.

In some embodiments, the server 10 can determine, based on the processed user data, physical characteristics of the user and determine the physical signature of the user based on the physical characteristics of the user. In some embodiments, the processed user data comprises physical characteristics data, and the server 10 can determine the physical characteristics of the user by comparing the physical characteristic data to stored physical characteristic data indicative or correlations between physical characteristics and physical characteristics data.

In some embodiments, the processed user data comprises physiological parameter data, such as heart rate data, blood pressure data, skin wetness data, or blood oxygen level data, and the server 10 can determine the one or more states of the one or more physiological parameters of the user by: comparing the physiological data to stored physiological parameter data indicative of correlations between states of physiological parameters and physiological parameter data.

In some embodiments, the server 10 can determine, based on the processed user data, a personality type of the user, and determine the emotional signature of the user based on the personality type of the user. In some embodiments, the processed user data comprises personality type data, and the server 10 can determine the personality type of the user by comparing the personality type data to stored personality type data indicative of correlations between personality types and personality type data.

In some embodiments, the processed user data comprises cognitive-affective competency data, and the server 10 can determine the one or more states of the one or more cognitive-affective competencies of the user by comparing the cognitive-affective competency data to stored cognitive-affective competency data indicative of correlations between states of cognitive-affective competencies and cognitive-affective competency data.

In some embodiments, the server 10 can determine at least one of: one or more mood states of the user, one or more attentional states of the user, one or more prosociality states of the user, one or more motivational states of the user, one or more reappraisal states of the user, and one or more insight states of the user. The server 10 can determine the one or more states of the one or more cognitive-affective competencies of the user based on the at least one of: the one or more mood states of the user, the one or more attentional states of the user, the one or more prosociality states of the user, the one or more motivational states of the user, the one or more reappraisal states of the user, and the one or more insight states of the user.

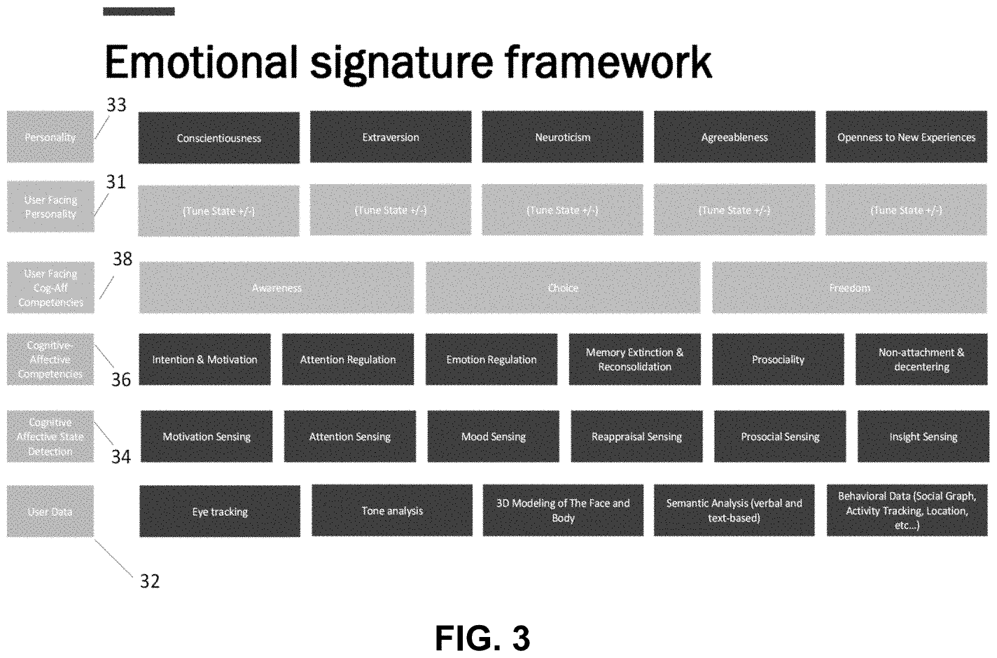

FIG. 3 shows an example relationship between different user data and how it relates to emotional signature metrics using cognitive-affective competencies and personality types. The framework can be stored at server 10 (e.g. in database 12) as code instructions and data records that map parameters for cognitive-affective competencies and personality types to user data and product variables. In row 32, there are shown different forms of techniques and analysis processes for determining user data relating to the user. In row 34, there are shown different types of cognitive-affective state detections methods, based on the type of user data that is captured. The server 10 can implement different types of cognitive-affective state detections methods, detects the type of user data that is captured, and selects the appropriate type of cognitive-affective state detections method for processing the user data. For example, eye tracking data may enable system 100 to sense a level of a user's attention, whereas 3D modelling and analysis of the user's face and body may enable system 100 to sense one or more moods of the user. In rows 36 and 38, there are shown different types of cognitive-affective competencies. In rows 31 and 33, there are shown different levels or states of different aspects of a user's personality type. The user data also provides different types of data to compute physical signatures for the users. There can be different components, dimensions and sub-dimensions for the physical signatures. Example physical signature components can be physical parameters (size, volume, weight, height); movement parameters (legs, arms, breasts, body); activity (high/low Intensity); preferences (how one wants to feel); sensory parameters (touch, taste, smell, sound, light. These break down to mechanistic components which differ for each person.

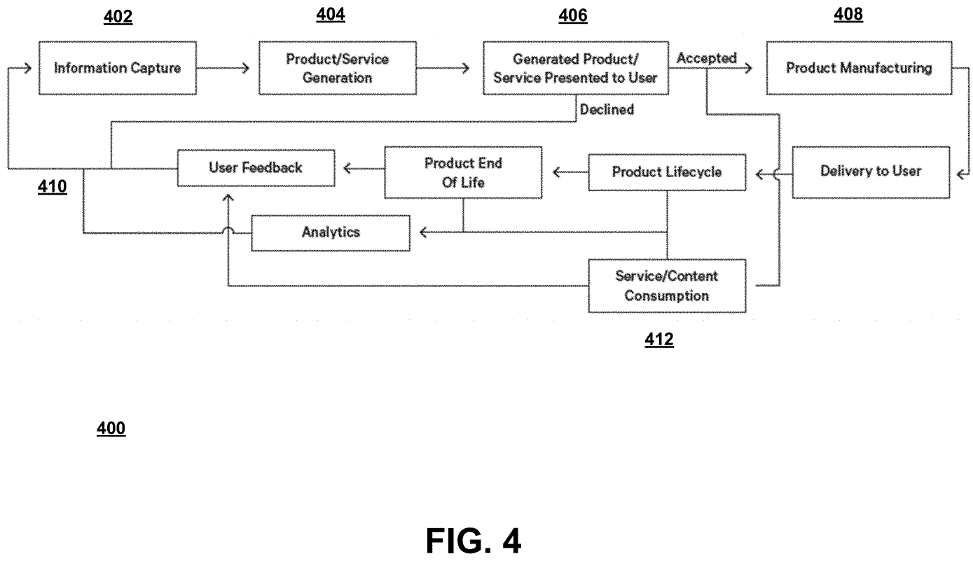

FIG. 4 shows a method 400 providing an interface 32 for product personalization.

The method 400 can be implemented by the server 10. For example, the server 10 can select ‘product’ category (apparel, hardware, software, content, service). The server 10 can quantify all variables of the products. Example variables include weight, stretch, colour, temp, time, environment, and so on. The server 10 can associate variables with user sensation states or feel states. For example, for products and services that involve taste, smell and colour are variables that can be associated with taste. The server 10 can take identified variables and define quantifiable targets based on the strongest determinant of a desired feel state. For example, Smell X and Colour Y=Sweet. The server 10 can take identified variables and further prioritise the variables based on which variables are required (via threshold parameters) to have unique quantified targets based on an users physical or emotional signature, which can correspond to values representing an individual's physical and emotional uniqueness and their optimal feel state. For example, Smell X can be the target for the general population, but Colour Y can be personalised for the individual in order to attain optimal feel state. As an example for the product or service that involves taste, the server 10 can compute taste metrics or values, such as for Person A: Smell X and Colour Y(0.7)=Sweet. The server 10 computes these product variables personalized for a user, which can be used as input data for the recommendation or creation process.

The server 10 can define quantifiable targets for different desired feel states. The server 10 can define quantifiable targets using different forms of statistical analyses (i.e. a principle component analysis) to identify the variables with most effect on feel state. These variables are then put through an experimental protocol whereby the server 10 changes the ‘quantifiable metric’ to see which quantity drives the sensation. There can be experimental data from labs captured as look up tables and logic gates. For example, the data may indicate that for the sensation of ‘comfortably warm’ the skin wetness variable is the highest rated variable. The server 10 can then test different percentages of skin wetness to find between 30-50% skin wetness is the quantified target to feel comfortable. Bra comfort variable is another example. The server 10 can define peak acceleration of the breast as an important variable and can define specific m/s² acceleration targets for different breast volumes.

As another example, specific HRV data can be associated with emotional sensations such as calm or stress. For example, in the case of a bra, the server 10 can define bra structure as underwire X+material modulus Y a determine whether a given bra structure meets the acceleration target and therefore provides values for the bra user's comfort sensation. As a further example, in the case of pants, the glute compression A and thigh compression B can provide a user with a sensation of confidence. As another example, server 10 can define Smell X and Light Colour Y as a calm sensation target.

The server 10 can further prioritise variables using unique quantified targets based on an individuals physical and emotional uniqueness and their optimal feel state. The server 10 can update the product variables and adjust the weighting for different target feel states. The order or the quantity of the variables can change individually. For example, variable Comfortable for Person 1 may be defined as needing a peak acceleration from 3-5 m/s² and person 2 may need a peak acceleration from 7-9 m/s². Each individual may also have unique needs for under band compression or other components of the bra. Instead of generically defining bra comfort to be peak acceleration of 5 m/s/s and a under band compression of 13 mmHg for all users, the server 10 can generate a personalized product (bra) that can customise the variable values or numbers for individuals. As another example, thermal comfort can be a unique variable for each user. Person 1 can have a specific need of 30% skin wetness and this can be combined with a specific material surface roughness or conduction metric to customize the product for the user. In a mindfulness space, the server 10 can normalise the range of HRV to match the ability of the user to manage stress. Person A with HRV x and person B with HRV y may both feel the same level of calm due to their innate ability to manage stress, by way of example.

At 402, user devices 16 capture user data for a user session over a time period. The server 10 can also capture user data from different sources, or predict user data using prediction models. For product recommendation or creation, the server 10 obtains the user data to measure the user or guest. The user data can be used to physically measure the user, and additional data can be captured by the server 10 or user devices 16 through prediction models. The server 10 can process the user data captured by the user devices 16 to extract personalisation metrics and test for them. For example, the user devices 16 can measure specific breast accelerations for a user. With prediction, the server 10 can use a data model, so the user devices 16 may not need to physically measure all aspects of the user but can still acquire data representative of these aspects of the user to generate the products. The server 10 can determine the known bra size, combined with the user's fit preference and activity for input into a prediction model to accurately predict breast acceleration.

The user data can be quantitative (e.g. physical measurements) or qualitative (e.g. perceptual preferences). The user data will be processed by generative models which will create a product or service to match the inputted user data. An interface 32 can display the output to receive input commands to modify or purchase/use the generated product (or service). If it is a product that is purchased, manufacturing instructions can be sent to the manufacturing queue 34, the product is made and delivered to the user. As noted, the term product as used herein extends to services, and if a service is selected, content can be electronically delivered to the user device 16. Once the user has been able to use the product or service, they are able to close the loop by providing feedback data the product/service which is transmitted and stored to increase the accuracy of the generative models. If the user received a product, it can be returned at the end of its life to be broken down to create new products.

For example, at 402, sensors can capture user data for a user session over a time period. The captured user data is processed at server 10 to extract user attributes. Server 10 stores and updates an attributable database 12 of product measurement records, movement features, perceptual preference features, physical signature features, emotional signature features, user records, and generative design models in memory. The server 10 can extract user attributes from the user data and associate with the product variables, the user attributes comprising measurement metrics, movement metrics, perceptual preference metrics, physical signature metrics, and emotional signature metrics.

The server 10 processes the user data of different data types to select different categories of products (or services) that are quantified by different product variables. For example, there can be different sub-categories of products or services identified to determine variables of the product. For example, the product can be a personalized bra or pants. Example services include video content and services for coaching and activities.

For a personalized bra, the server 10 captures movement data and feel preferences as input data for generative design models to create a digital artwork or pattern to be laser cut, applied or screen printed, or other design process to give specific mechanical properties to achieve movement management and desired feel state personalized for the user. The server 10 can generate associated files for automating assembly and manufacturing of the product. The product is assembled and shipped to the user. The movement data can relate to parts of the users body, such as breast movement data.

For a personalized pair of pants, the server 10 captures leg movement data, including movement variability and gait, and user preferences for the sensation of balance, speed, and flow as input data for generative design models to create a digital artwork or pattern to be laser cut, applied or screen printed, or other design process to give specific mechanical properties to achieve movement management and desired feel state personalized for the user. The sever 10 can generate associated files for automating assembly and manufacturing of the product. The product is assembled and shipped to the user.

For personalized service content, an example is an exercise and nutrition plan that involve different types of content. The server 10 can capture measurements, activity preferences, dietary restrictions, and so on, by user device 16. The server 10 can input this data into generative models to generate exercise content assembled to match inputs. Meal plan menu can be generated to match inputs and the user can be served scheduled content to follow, and sent meal plan or meal kits according to the generated menu.

The server 10 uses the physical signature metrics and emotional signature metrics to generate the product or service. For example, the server 10 serves the user content and products (journals, meditation equipment) based on personality trait values that can be computed by user data captured along a personalized emotional fitness journey. The server 10 categorizes the physical signature data, emotional signature data, or social signature data to map to different variables for products or services. For example, the server 10 can process user data to determine that the user needs support dealing with anxiety, and content and products to aid in controlling anxiety are delivered. As an example for physical signature data, the server 10 can process user data to determine that the user has low flexibility, and content and products aimed towards improving flexibility are delivered. As an example for social signature data, the server 10 can process user data to determine that the user is introverted, and the server 10 can generate products that serve user more internal reflection-based content. The server 10 can process user data to determine that the user is extroverted and the server 10 can generate products that serve user group classes or group discussion-based content.

The methods 400 can involve, for example, a user providing credentials to the user device 16 at user interface 24 to trigger product personalization and real-time data capture to improve their general physical or emotional wellbeing at a current time period based on the real-time user data. For example, the user activates on user device 16 an emotional wellbeing application (not shown) stored on memory 20 to trigger the user interface 24. The emotional wellbeing application invites the user to input user data to user device 16. The user device 16 receives the user data relating to the user from the user interface 24, which can be collected from different sensors 24, 26, 28 in real-time to provide input data for generating products based on (near) real-time computation of the emotional wellbeing metrics based on the real-time data. For example, in response to activating emotional wellbeing application, the user may be prompted to complete a series of exercises and/or questionnaires, and the user interface 24 collects real-time user data throughout the series of exercises or other prompts. For example, a questionnaire may be presented to the user on user interface 24 and may require the user to answer one or more questions comprised in the questionnaire. Alternatively, the user may be prompted to speak out loud to discuss emotionally difficult events or how they feel about others in their life. The user interface 24 can collect the captured audio data for provision to the server 12. In other examples, with consent data obtained from the user interface 24, various forms of biometric data may be passively recorded throughout the user's day-to-day life as captured from different sensors 24, 26, 28 in real-time. Additionally, non-biometric data may also be recorded at user device 16, such as location data relating to the user. Such data may be processed to detect and quantify changes in levels of cognitive-affective competencies, and any other information used to measure the user's emotional signature, as described in further detail below.

The user may provide the answers, for example, via text input to user interface 24, or alternatively may speak the answers. Spoken answers may be detected by microphone 28 and utterances can be converted into audio data by processor 22. Prior to, during, or after the completion of the questionnaire, the emotional wellbeing application may send control commands to cause camera 26 to record images and/or video of the user. The images may comprise at least a portion of the user's body, at least a portion of the user's face, or a combination of at least a portion of the user's body and at least a portion of the user's face. The captured images are then converted into image data (which may comprise video data), which forms part of the overall user data that is received at user device 16.

The combination of audio data, text data, and image data, and any other data input to user device 16 and that relates to the user, may be referred to hereinafter as user data. Other suitable forms of data may be comprised in the user data. For example, the user data may comprise other observable data collected through one or more Internet of Things devices, social network data obtained through social network analysis, GPS or other location data, activity data (such as steps), heart rate data, heart rate variability data, data indicative of a duration of time spent using the user device or one or more specific applications on the user device, data indicative of a reaction time to notifications appearing on the user device, social graph data, phone log data, and call recipient data.

The server 10 can store the user data in records indexed by an identifier for the user, for example. The user device 16 can transmit captured user data to the server 10 for storage in database 12. In some embodiments, the user device 16 can pre-process the user data using the emotional wellbeing application before transmission to server 16. The pre-processing by the emotional wellbeing application can involve feature extraction from raw data, for example. The user device 16 can transmit the extracted features to server 10, instead of or in addition to the raw data, for example. The extracted features may be facilitate efficient transmission and reduce the amount of data transmitted between the user device 16 and server 10, for example.

According to some embodiments, in addition to user data being captured via the sensors 24, 26, 28 of user device 16, wearable sensors (e.g. a heart rate monitor, a blood pressure sensor) positioned on the user may provide additional data (such as the user's physical activity levels) and may be inputted to user device 16 and may form part of the user data received at user device 16.

At 404, in response to receiving a product request from the interface 32, the server 10 generates a product and associated code files. For example, the server 10 generates a product and associated code files by processing the extracted user attributes and the target parameters for a target feel state using the generative design models and the attributable database. As part of product generation, the server 10 selects a product category and product variables. For example, the product can be a personalized bra and the associated code files can be pattern and material files generated by a 3D modelling system coupled to or in communication with the server 10.

For product generation, an existing product or service type can be identified from a table and then customized for a user. The selected product can be used for personalization of the product based on the user data. The product type can be selected using the physical or emotional signature of the user, for example. The components of the product are identified and personalized for the user data. The components of the product are assembled to generate the product. The server 10 can encode which components can fit together to generate the product. Multiple users generate the same product type with personalized components based on the user data.

The server 10 can generate BOM files automatically for the product. The server 10 can generate a list of component to create the product.

The server 10 can generate customized content and files for assembly instructions. Inputs are taken (e.g. time, activity preference, equipment preference, physical signature, emotional signature) and can be combined with pre-captured videos to create the output content. The server 10 can generate the content on demand with wholly virtual facilitators and instructors based on the inputs. The server 10 can identify and arrange sub-components of the content as part of product generation on demand.

At 406, interface 32 displays a visualization of the product with a selectable purchase option. In response, the interface 32 receives purchase instructions for the product in response to selection of the selectable purchase option displayed with the visualization of the product. The server 10 can generate data for the visualization of the product using presentation operations.

The server 10 can generate the visualization of the product generated by the design model. There can be encoded instructions for the product, and a rendering engine can display the product visualization as a part of the system interface 32. The visualization data is linked to the product and generative models by the backend server. For a Bra example, movement data from the breast sensor is captured and used to create a motion intervention in the product, and the visualization can have motion traces (“butterfly”) visualized to show the user the influence their data is having on the product. As another example, a visualization of the user's balance can show them why they should take a recommended class to assist in improving their overall balance.

The server 10 can generate custom manufacturing instructions as part of the product generation process. The server 10 can convert images to knitting instructions, 3D printing instructions, laser cutting instructions, and encode the manufacturing data in associated files.

At 408, the server 10 triggers a product manufacturing process in response to receiving the purchase instructions for the product. The server 10 transmits manufacturing instructions for the product to a manufacturing queue 34 to trigger production. The system 100 can coordinate delivery of the product. Over the product lifecycle to product end of life, the server 10 can receive user feedback. As noted, a product can also include content delivered by a content platform or service. The server 10 can receive data regarding consumption of the content by the user as feedback.

At 410, the server 10 receives user feedback from different sources over the product lifecycle and updates its database 12. The server 10 updates generative design models using the feedback.

In some embodiments, the product is a service that involves content. At 412, the server 10 generates content and delivers content to the user, at interface 32 or to another device.

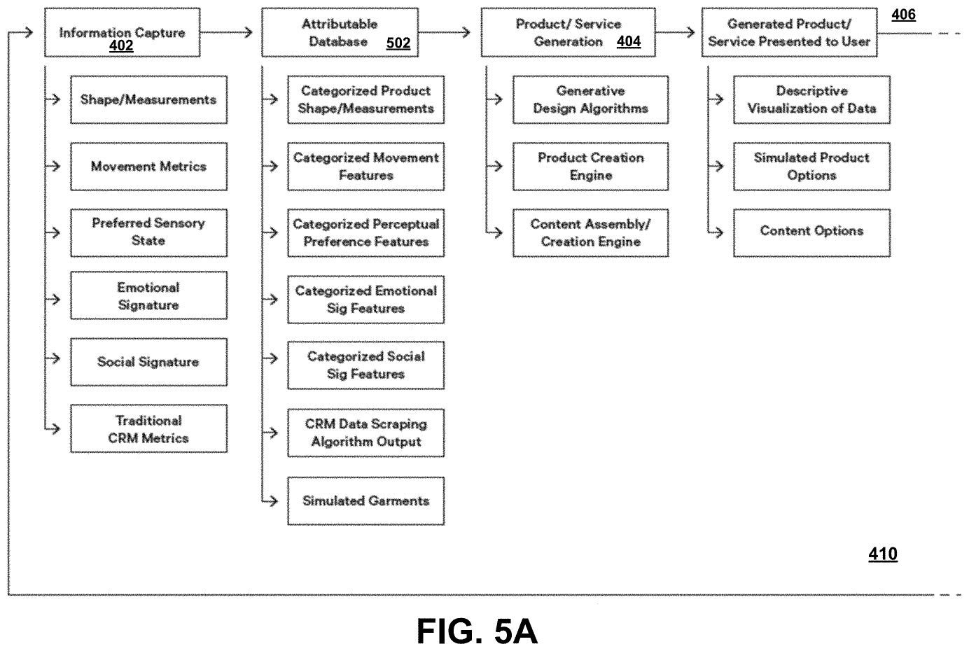

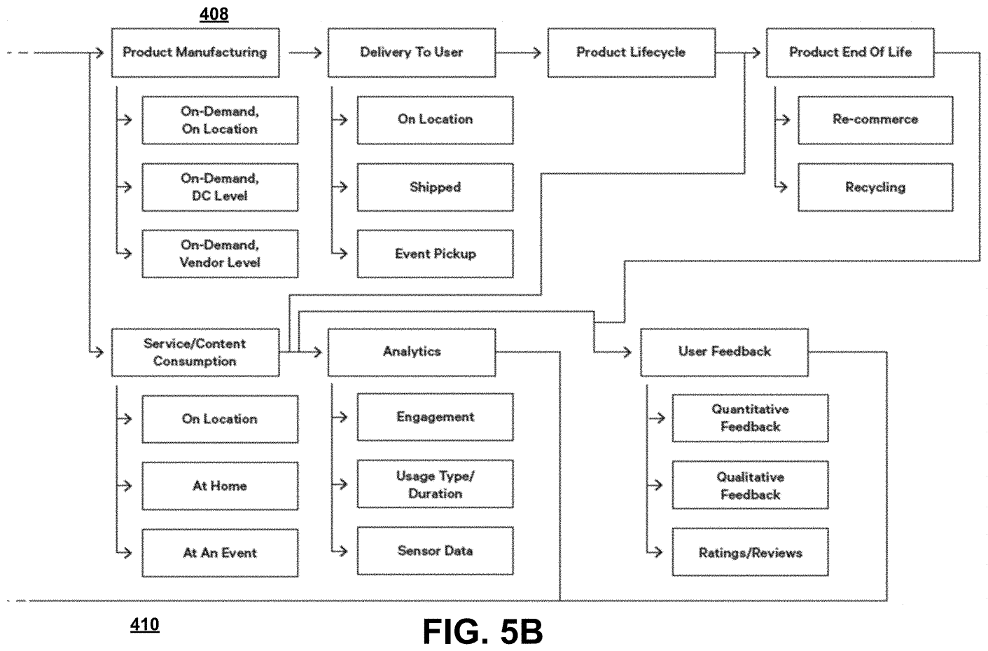

FIGS. 5A and 5B shows operations of the method 400 of providing an interface for product personalization in further detail. The operations of 402, 404, 406, 408, 410 can be implemented as described in relation to FIGS. 4, 5A, 5B, for example.

As noted, at 402, user devices 16 capture user data for a user session over a time period. Different capture technology can be used to capture user data for a user session over a time period. The captured user data is processed at server 10 to extract user attributes or metrics. For example, user attributes can include shape/measurement metrics, movement metrics, perceptual preference metrics, emotional signature metrics, social signature metrics, traditional CRM metrics, preferred sensory state metrics, equipment metrics, and so on. The server 10 can extract different user attributes from the user data and associate these metrics with the product variables. The metrics can be associated with product variables by predefined look up tables or logic gates of server 10, for example.

The user device 16 transmits, over network 14, the user data to servers 10. Servers 10 then process the user data using different processing techniques. For example, servers 10 may process the image data using any of various facial and/or body analysis or modelling techniques known in the art or yet to be discovered. In addition, servers 10 may process voice data using any of voice analysis techniques (including tone analysis techniques) known in the art or yet to be discovered. In addition, servers 10 may process user input data (which may include audio data or text data) using different voice, text, social network, or behavioural analysis techniques (including tone analysis techniques and semantic analysis techniques) to extract features or metrics that can be used to compute a real-time emotional signature for the user. The real-time emotional signature can be used for product generation (or recommendations) displayed as visualizations via interface 32 or user interface 24 of user device 16. Servers 10 may process user input data (which may include audio data or text data) using different voice, text, social network, or behavioural analysis techniques (including tone analysis techniques and semantic analysis techniques) to extract features or metrics that can be used to compute a real-time physical signature for the user.

By processing the user data in this fashion, servers 10 are able to identify one or more mood levels or states of the user. In addition to mood sensing (e.g. determining the user's current mood state), servers 10 are able to perform operations to compute different metrics corresponding to attention sensing (e.g. determining the user's external attentional deployment, internal attentional deployment, etc.), prosocial sensing (e.g. determining the user's emotional expression and behaviour with others, etc.) motivational state sensing, reappraisal state sensing, and insight state sensing. Such sensing techniques are examples of state detection sensing techniques that may be used to quantify an individual user's levels of cognitive-affective competencies, as well as determine the individual's personality type based on collected data.

For example, metrics or data corresponding to attention sensing may be determined by processing eye tracking data and through 3D modelling of the user's face and/or body, as well as the context or environment in which the user is in, and in addition to the object of attention or lack of such an object. Prosociality sensing relates to the detection of a user's positively/negatively valenced actions towards another person or towards themselves (e.g. giving a compliment, transmitting a positive/negative emotion such as smiling, mentioning a positive/negative action another person has undertaken, etc.).

Motivational sensing relates to computation of metrics based on the detection and distinction of the two subsystems of motivation known as the approach and avoid systems, which guide user behaviour based usually on a reward or a punishment (e.g. identifying a user's motivation through the way they describe their reason for completing a task, specific emotions displayed during a goal-oriented behaviour, etc.). Such motivation may be determined by processing the user's data input and activity data.

Reappraisal state sensing relates to computation of metrics based on detection of the user's recollection of an event and its affective associations, such associations being simultaneously weakened through active or passive means (e.g. having a user recall a difficult event over time and monitoring changes in emotional expression during the recollection). Extinction and reconsolidation can depend on numerous factors, such as level-of-processing, emotional salience, the amount of attention paid to a stimulus, the expectations at encoding regarding how memory will be assessed later, or the reconsolidation-mediated strengthening of memory trace. Extinction does not erase the original association, but is a process of novel learning that occurs when a memory (explicit or implicit) is retrieved and the constellation of conditioned stimuli that were previously conditioned to elicit a particular behavior or set of behavioral responses is temporarily labile and the associations with each other are weakened through active or passive means. Such recollection may be determined by processing the user's data input, biometric data, and historic physical and emotional signatures and associated recommendations.

Insight sensing relates to the computation of metrics based on realization of “no-self” or non-attachment, which is a distinction between the phenomenological experience of oneself and one's thoughts, emotions, and feelings that appear “thing-like” and is described as a “release from mental fixations”. Insight sensing also relates to decentering which introduces a “space between one's perception and response” allowing the individual to disengage or “step outside” one's immediate experience in an observer perspective for insight and analysis of one's habitual patterns of emotion and behavior. Insight sensing may detect moments when an individual is relating to their thoughts, feelings, emotions, or bodily sensations as separate from who they are through how they are describing their experience and other non-verbal cues. Such sensing may be determined by processing the user's data input, biometric data, and historic physical and emotional signatures and associated recommendations.

A personality type of the user may be generally estimated by metrics that correspond to values for one or more states or levels of any of various different models of personality types, such as the five-factor model: openness/intellect, conscientiousness, extraversion, agreeableness, and neuroticism/emotional stability. A mood state of the user may be determined by computed metrics that include one or more indications of: amusement, anger, awe, boredom, confusion, contempt, contentment, coyness, desire, disgust, embarrassment, fear, gratitude, happiness, interest, love, pain, pride, relief, sadness, shame, surprise, sympathy, and triumph. Cognitive-affective competencies of the user may include one or more of: intention and motivation, attention regulation, emotion regulation, memory extinction and reconsolidation, prosociality, and non-attachment and decentering.

The automated detection/recognition of emotional characteristics in a person can be determined by processing the user data to extract and evaluate features relevant to emotional characteristics from the user data.

Based on the determined personality type and states of cognitive-affective competencies of the user, an emotional signature of the user is determined by the server 10 using data received from the user device 16. According to some embodiments, the emotional signature is a combination of the data values corresponding to the determined personality type and states of cognitive-affective competencies of the user by the server 10 access the user data stored in databases 12 as captured by the user device 16 in (near) real-time. The emotional signature may act as a unique metric (or combinations of metrics) identifying the current overall emotional wellbeing of the user.

At 502, server 10 stores and updates the attributable database 12 in memory with categorized metrics. The server 10 processes the shape and measurement metrics, movement metrics, perceptual preference metrics, physical signature metrics, emotional signature metrics, social signature metrics, traditional CRM metrics, preferred sensory state metrics, and equipment metrics to update the attributable database 12. The server 10 generates categorized features corresponding to the shape and measurement metrics, movement metrics, perceptual preference metrics, emotional signature metrics, social signature metrics, traditional CRM metrics, preferred sensory state metrics, and equipment metrics.

The server 10 can convert the user data into features for the attributable database 12. As an example for measurement data, the server 10 maps the right bicep measurement under the right bicep attribute in the attributable database 12. There may be some machine learning or classification using Natural Language Processing (NLP) to tag and classify open text inputs.

At 404, the server 10 generates a product and associated code files. For example, the server 10 generates a product and associated code files by processing the extracted user attributes and the target parameters for a target feel state using the generative design models and the attributable database. As part of product generation, the server 10 selects a product category and product variables. The server 10 selects a generative design model for product generation. The associated code files can include bills of materials (BOM) files and the generative design model can involve generating the BOM files. The associated code files can include content assembly files and the generative design model can involve generating the content assembly files.

Example generative design models include models for 3D modeling software or plug-in to generate geometric representations for products as part of the product personalization. Example generative design models include soft body simulations. The generative design models can also generate the BOM files and content assembly instructions. The generated product can include BOM files and the content assembly instructions. The manufacturing queue 34 can use the BOM files and the content assembly instructions to manufacture the personalized product.

At 406, interface 32 displays a visualization of the product with a selectable purchase option. The server 10 can generate the product and associated code files that includes a descriptive visualization of data as instructions for the interface 32 to display the visualization of the product. The server 10 can also generate simulated product options. The associated code files can include educational services and content. The associated code files can include generated content to support improvements to practices or habits. The content can include physical activities, for example.

In an example embodiment, the product is a garment. The attributable database 502 can have simulated garment records. At 406, the hardware server 10 generates simulated product options as part of the product and associated code files. The interface 32 displays a visualization of the simulated product options. The simulated product options can include soft body physics simulation, hard body physics simulation, static 3D viewer and AR/VR experience content.

In response, the interface 32 receives purchase instructions for the product in response to selection of the selectable purchase option displayed with the visualization of the product.

At 408, the server 10 triggers a product manufacturing process in response to receiving the purchase instructions for the product. The server 10 can support on-demand and on-location product manufacturing. The server 10 can support on-demand manufacturing at a DC-level or vendor-level, for example.

The system 10 can trigger instructions for delivery of the product to a user, and tracking of data for the delivery process. The tracking data can include location data, shipping data, and event pickup data.

The server 10 transmits manufacturing instructions for the product to a manufacturing queue 34 to trigger production. The system 100 can coordinate delivery of the product. The system 100 can monitor the product lifecycle to receive data about the product until its end of life. The data can relate to re-commerce or recycling of the product. The data for recycling of the product can include data for polymer breakdown, biodegradation, polymer buildup, biological production, and so on.

The server 10 can receive different types of feedback about the product. As noted, a product can also include content delivered by a content platform or service. The server 10 can receive data regarding consumption of the content by the user as feedback. The consumption data can include location data, home data, and event data. The data can also be analytics data about engagement with the product, usage type data and duration, sensor data, and data indicating whether the product was shared with other users. The data can also be review data and testing data about the product.

At 410, the server 10 receives user feedback and updates generative design models using the feedback data. The server 10 improves the generative design models over time using the feedback data. For example, users may provide feedback data indicating that they were not satisfied with a recommended product. The server may use this feedback data to re-define the user's preferred range of product attributes in the generate design model in order to better align with the user's preferences.

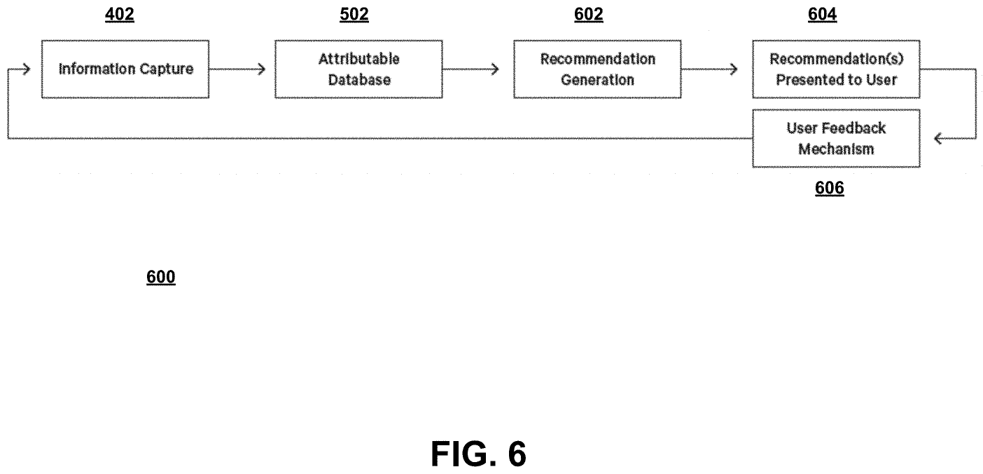

FIG. 6 shows a method 600 providing an interface for product recommendations. The method 600 can involve operations similar to the operations of the method 400 for generating products. The operations of 402 and 502 can be implemented as described in relation to FIGS. 4, 5A, 5B, for example. The server 10 implements method 600 for providing an interface 32 with product recommendations.

The user data can be quantitative (e.g. physical measurements, biometric data) or qualitative (e.g. perceptual preferences). The user data is processed by the server 10 (with recommendation engine) which will query an attributed database of all products and services to shortlist those that best match the inputs. Shortlisted items from the database will be presented for the user to select from. Once a product has been selected and trialed by the user, they are able to close the loop by providing feedback data on how well the recommendation lined up with their needs. The feedback is stored and used to increase the accuracy of the recommendation engine.

As noted, at 402, user devices 16 capture user data for a user session over a time period. For example, hardware processor 18 can be programmed with executable instructions for an interface to obtain user data for a user session over a time period. The processor 18 can transmit a product request for the user session. The interface 32 provides product recommendations for the user session in response to the product request, and receives a selected product of the product recommendations. The interface 32 can receive feedback data on the selected product. The user device 16 has one or more sensors for capturing the user data during the time period. The user device 16 has a transmitter for transmitting the captured user data to the interface 32 of the hardware processor 18 or the hardware server 10 over the network to compute the product recommendations.

At 502, the hardware server 10 couples to the memory to access the attributable database. The server 10 stores and updates the attributable database 12 in memory with categorized metrics. The server processes shape/measurement metrics, movement metrics, perceptual preference metrics, physical signature metrics, emotional signature metrics, social signature metrics, traditional CRM metrics, preferred sensory state metrics, and equipment metrics to generate categorized features.

At 602, server 10 generates product recommendations. The server 10, in response to receiving the product request from the interface 32, selects product category and product variables. The server 10 computes target parameters for a target feel state for the user using the extracted user attributes.

The server 10 computes product recommendations using a recommendation engine to process the extracted user attributes and the target parameters for the target feel state.

In some examples, instead of creating a product from components based on the inputs, the server 10 can use a recommendation engine for searching the database 12 of products to find best matches to the inputs for the user A recommended product can then be personalized, using the method 400 described herein. The server 10 can implement intermediary steps between recommendation and full personalization.

There can be different features extracted from the user data for product recommendation. Not all data that is captured is necessary for each product/service recommendation, but different products or services can access the different datasets for generating a product or service recommendation.

The recommended products can be displayed within the interface 32 for selection. They can be in 3D display with movement such as rotating on a turntable.

At 604, the server 10 transmits the product recommendations to the interface 32 over a network. In an example embodiment, the product is a garment. The attributable database 502 can have simulated garment records. At 604, the hardware server 10 generates simulated product options as part of the recommended product and associated code files. The interface 32 displays a visualization of the simulated product recommendations. The simulated product options can include soft body physics simulation, hard body physics simulation, static 3D viewer and AR/VR experience content.

The interface 32 displays the product recommendations. The server 10 receives notification of a selected product from the interface 32.

At 606, the server 10 receives feedback data on the selected product. The server 10 can update the recommendation engine based on the feedback data on the selected product. The server 10 can also update the information capture process using the feedback data.

In some embodiments, the user attributes include quantitative user attributes of physical metrics and qualitative user attributes. The qualitative user attributes include the emotional signature metrics of the user.

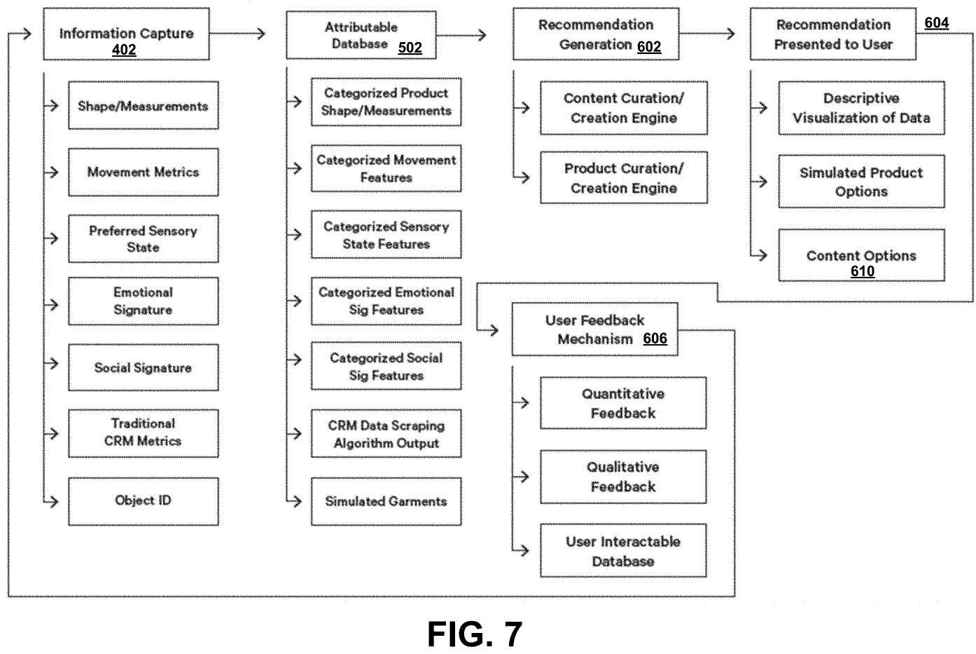

FIG. 7 shows operations of the method 600 of providing an interface for product personalization in further detail. The operations of 402, 502, 602, 604, 606 can be implemented as described in relation to FIGS. 4, 5A, 5B, 6 for example.

At 402, user devices 16 capture user data for a user session over a time period. The captured user data is processed at server 10 to extract user attributes or metrics. For example, user attributes can include shape/measurement metrics, movement metrics, perceptual preference metrics, emotional signature metrics, social signature metrics, traditional CRM metrics, preferred sensory state metrics, equipment metrics, and so on. The server 10 can extract different user attributes from the user data and associate these metrics with the product variables.

At 502, server 10 stores and updates the attributable database 12 in memory with categorized metrics. The server processes the shape/measurement metrics, movement metrics, perceptual preference metrics, physical signature metrics, emotional signature metrics, social signature metrics, traditional CRM metrics, preferred sensory state metrics, and equipment metrics to generate categorized features.

At 602, server 10 generates product recommendations. The server 10, in response to receiving the product request from the interface 32, selects product category and product variables. The server 10 computes target parameters for a target feel state for the user using the extracted user attributes. For example, the server 10 generates a product recommendation by processing the extracted user attributes and the target parameters for a target feel state using the generative design models and the attributable database. As part of product recommendation, the server 10 selects a product category and product variables.

In some embodiments, generating the product recommendation involves the server 10 generating a product and associated code files. The associated code files can include BOM files and the generative design model can involve generating the BOM files. The associated code files can include content assembly files and the generative design model can involve generating the content assembly files.

At 604, the server 10 transmits the product recommendation to the interface 32 to display a visualization of the product recommendation with a selectable purchase option. The server 10 can generate the product recommendation that includes a descriptive visualization of the recommended product(s) as instructions for the interface 32 to display the visualization of the product recommendation. The server 10 can also generate simulated product options. The server 10 can also generate associated content such as educational services and practices. The product recommendation can include generated content to support improvements to practices or habits. The content can include physical activities, for example. The interface 32 can provide a selectable purchase option for the recommended product.

The interface 32 receives purchase instructions for the product in response to selection of the selectable purchase option displayed with the visualization of the product.

At 606, the server 10 receives user feedback on the product recommendation and updates the recommendation engine using the feedback data. The server 10 improves the recommendation engine over time using the feedback data. The feedback data can be review data and testing data about the product.

The method 600 for providing an interface for product recommendations can involve similar operations to the method 400 providing an interface for product personalization. In some embodiments, the method 600 involves generating product recommendations for a user based on their physical or emotional signature, and providing the recommendations via an interface 32. In some embodiments, the method 400 involves generating products for a user based on their physical or emotional signature, and providing visualizations of the generated products via an interface 32.

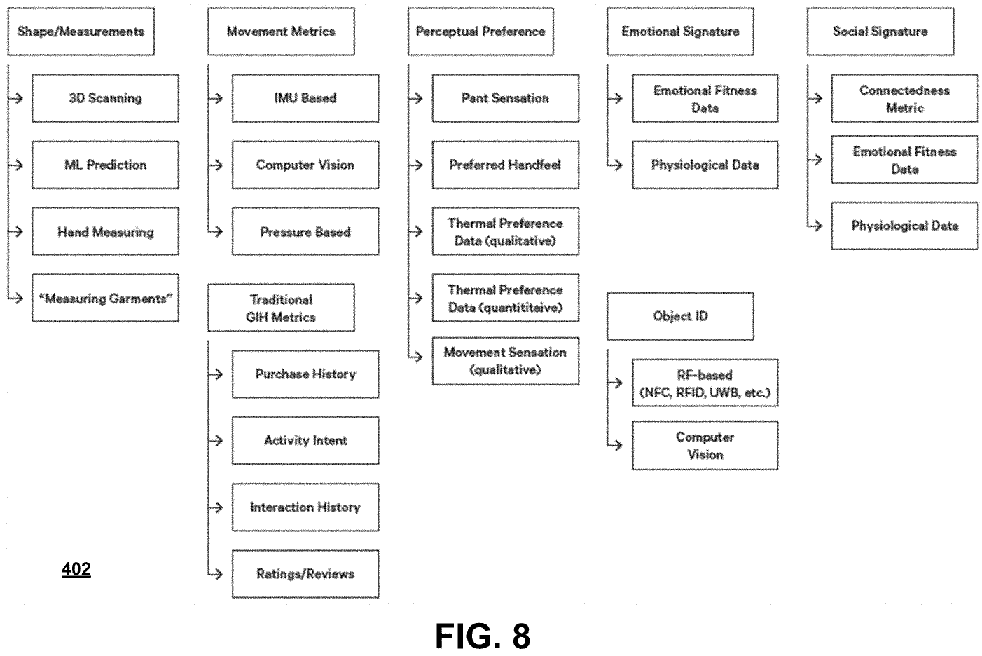

FIG. 8 shows example operations of capturing user data in further detail. As noted, at 402, user devices 16 capture user data for a user session over a time period. Different capture technology can be used to capture different types of user data for a user session over a time period. Different types of user devices 16 can be used to implement different capture technology and to capture different types of user data. The server 10 can process the user data to generate products in some embodiments. The server 10 can process the user data to recommend products in some embodiment. The recommended products can also be generated by the server 10, for example.

The captured user data can include shape/measurement data, movement metrics, perceptual preference data, physical signature data, emotional signature data, social signature data, resonance data, connectedness data, CRM data, equipment metrics, and so on. The server 10 can extract different user attributes from the user data and associate these metrics with product variables as part of the product generation.

The shape/measurement data can be captured by different capture technology such as 3D scanning, machine learning prediction shape or measurement outputs, hand measuring data, garment measuring data, and so on.

The movement metrics can be captured by different capture technology such as inertial measurement units based devices, computer vision devices, pressure based devices, and so on. The perceptual preference data can include different types of garment preference data, such as pant sensation data, preferred handfeel data, thermal preference data (qualitative or quantitative), and movement sensation data (qualitative). The physical signature data can include different types of physiological data which can include different types of quantitative or qualitative data such as preference data (e.g. preferred types of exercise), goals data, strength and weakness data, medical history data (e.g. past injuries), physiological data (e.g. blood pressure) and so on, The emotional signature data can include different types of qualitative or quantitative data such as personality data, mood state data, emotional fitness data, personal values data, goals data, strength and weakness data (e.g. wheel of life data), physiological data (HRV, breathing), and so on. The CRM data can include purchase history data, activity intent data, interaction history data, review data, and so on. The equipment data can be collected using radio frequency technology, such as NFC, RFID, UWB, and so on.

The server 10 can derive biodata through computer vision, such as heart rate variability by processing videos of face using photoplethysmography imaging. The server 10 can derive metrics by understanding details related to categorizations of body language features or emotions.

The captured user data is processed at server 10 to extract user attributes or metrics. For example, user attributes can include shape/measurement metrics, movement metrics, perceptual preference metrics, physical signature metrics, emotional signature metrics, social signature metrics, traditional CRM metrics, preferred sensory state metrics, equipment metrics, and so on. The server 10 can extract different user attributes from the user data and associate these metrics with the product variables.

The user device 16 transmits, over network 14, the user data to servers 10. Servers 10 then process the user data using different processing techniques. For example, servers 10 may process the image data using any of various facial and/or body analysis or modelling techniques known in the art or yet to be discovered. In addition, servers 10 may process voice data using any of voice analysis techniques (including tone analysis techniques) known in the art or yet to be discovered. In addition, servers 10 may process user input data (which may include audio data or text data) using different voice, text, social network, or behavioural analysis techniques (including tone analysis techniques and semantic analysis techniques) to extract features or metrics that can be used to compute a real-time emotional signature for the user. The server 10 can compute the physical signature data using voice, text, and social network data. The real-time emotional signature can be used for product generation (or recommendations) displayed as visualizations via interface 32 or user interface 24 of user device 16.

The server 10 can generate products in some embodiments. Further, the recommended products can also be generated by the server 10, for example.

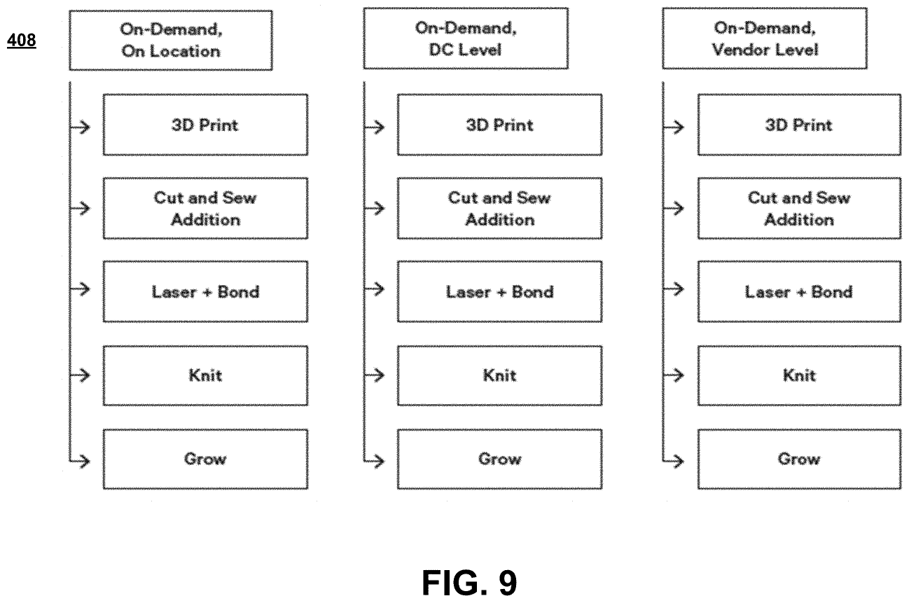

FIG. 9 shows example operations for a product manufacturing process in further detail. As noted, at 408, the server 10 triggers a product manufacturing process in response to receiving the purchase instructions for a product. As shown, the server 10 can support on-demand and on-location product manufacturing. The server 10 can support on-demand manufacturing at a DC-level or vendor-level, for example. The product manufacturing process can involve different operations such as 3D printing, cutting and sewing, laser and bond, knit, growing biological materials. The product manufacturing process can involve job preparation and queuing data.

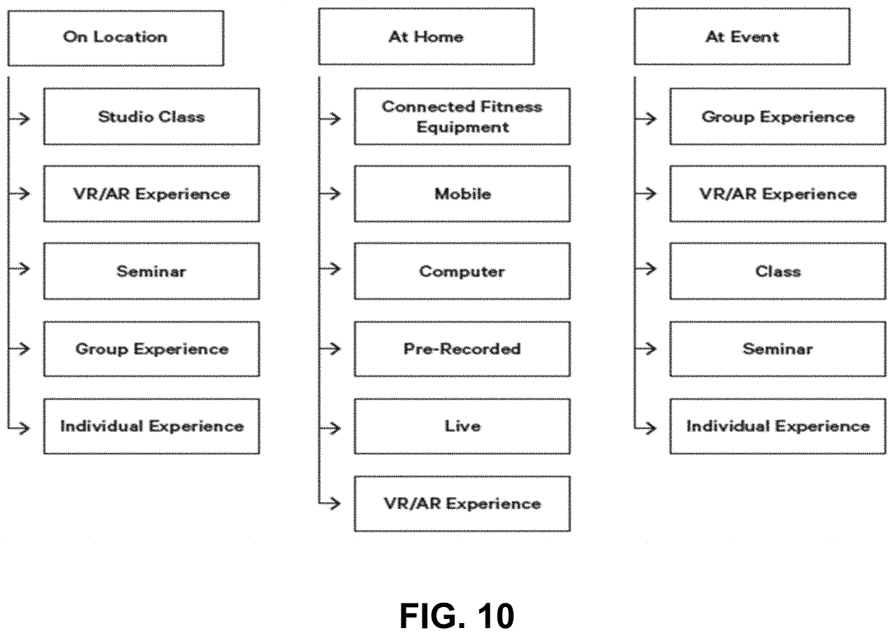

In some embodiments, the product is a service that involves content. At 412, the server 10 generates content and delivers content to the user. FIG. 10 shows example operations for content generation and delivery in further detail. The content delivery can be on-location, at home, or at an event. The content can be delivered to interface 32 or another device, such as an immersive hardware device. The content can be for a group experience or an individual experience.

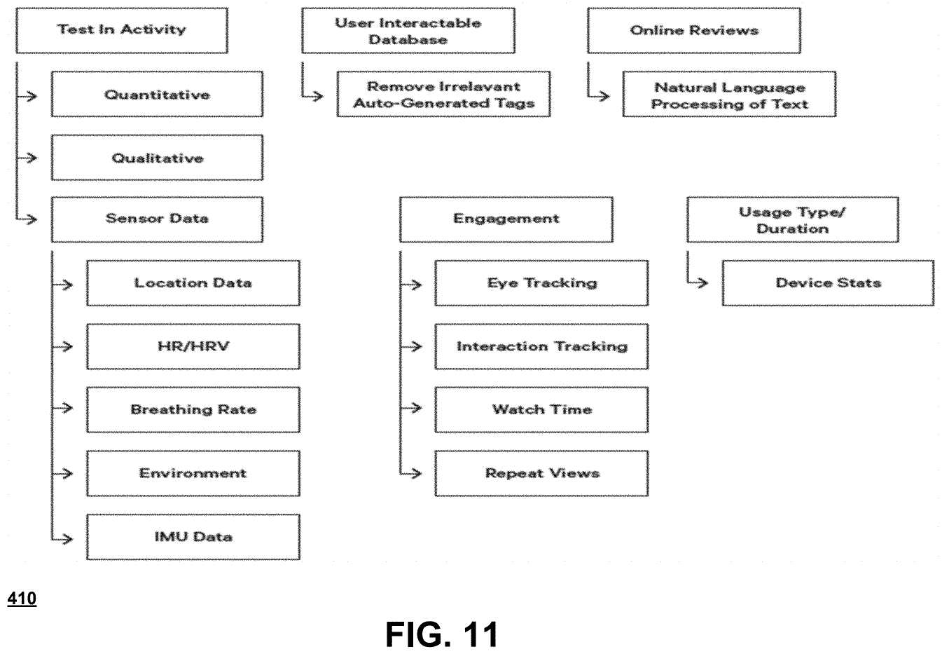

FIG. 11 shows example operations for feedback data in further detail. At 410, the server 10 receives user feedback from different sources over the product lifecycle and updates its database 12. The server 10 updates generative design models using the feedback. The feedback can be collected during an activity that involves the product. The feedback data can be extracted from an interactive database, which can involve removing tags from user content. The feedback data can be extracted from review data using natural language processing of text. The feedback data can be from engagement data captured as user data such as eye tracking data in relation to content, interaction tracking data, watch time for content, repeat views for content, and so on. The feedback data can be from user type and duration data from device or equipment statistics. The feedback data can be from sensor data, such as location data.

The servers 10 generate one or more personalized products or recommendations in response to computing data for the physical or emotional signature of the user. The servers 10 can generate personalized products or product recommendations. For example, a recommendation may comprise a recommendation to access or use particular content, coaches, events or other experiences for improving the user's physical signature. For example, a physical signature may indicate that the user is having difficulty engaging in cardiovascular exercise for extended periods of time due to low oxygen intake. In response, the product recommendations may include consuming content (e.g. video, one-on-one personal training) aimed at improving cardiovascular endurance and VO2 max. As another example, a recommendation may comprise a recommendation to access or use particular content, coaches, events, groups, platonic/romantic matches, or other social or emotional learning experiences, for improving the user's emotional signature. For example, an emotional signature may indicate that the user is having difficulty in disrupting negative mental rumination as a result of low levels of decentering and non-attachment. In response, the product recommendations may include consuming content (e.g. video, audio, one-on-one therapy) aimed at teaching a particular meditation which focuses on decentering.

The personalized products or product recommendations generated by system 100 and outputted to the interface 32 may take the form of a training program to be executed by the user. For example, the training program may comprise one or more microcycle phases (daily-weekly programming), one or more mesocycle phases (2-6 week programming), and one or more macrocycle phases (annual programming). The intensity and volume of the training sessions may be varied linearly or non-linearly. While the levels of cognitive-affective competencies may vary over time, they are generally trainable. Thus, through repeated interventions (e.g. meditation), the propensity for a person to process, for example, emotional stimuli in a negative or positive way may change based on training duration and consistency.

The personalized products or product recommendations generated by system 100 and outputted to the interface 32 may take the form of recommendations based on the user's physical fitness and skill level. For example, a user interested in yoga classes may receive, based on having little history of similar physical exercise, recommendations for beginner classes that are more appropriate for their skill level than intermediate or advanced classes. As the user's physical fitness and skill improve, the system 100 may recommend more challenging classes.

The personalized products or product recommendations generated by system 100 and outputted to interface 32 may take the form of recommendations based on the user's desired feel state outcome. For example, a user expressing a desire to feel energized may receive recommendations for high-energy physical activity while a user expressing a desire to feel calm may receive recommendations for guided meditations designed to relax and calm the mind.

The system 100 can store data for product classes or product recommendations in database 12 and server 10, and generate the personalized products or recommendations by identifying one or more product recommendations from the stored data. For example, the recommendations may be generated based on known recommendations stored in association with known personality types and states of cognitive-affective competencies or based on known recommendations stored in association with known physical characteristics. Such associations may be stored, for example, in databases 12, and may be accessed by servers 10.

Over time, through repeated collection of user data, the physical and/or emotional signature of the user may be tracked or monitored by server 10. The system 100 will continue to receive user data in real-time from the user device 16 to re-compute the physical and/or emotional signature of the user based on the updated user data. The system 100 can continuously collect user data and re-compute the physical and/or emotional signature. For example, after generating personalized products or product recommendations, the user may repeatedly or regularly interface with the user device 16 to obtain or capture additional user data that is used to compute an updated physical or emotional signature. The updated physical or emotional signature may be compared by the server 10 to the last known or computed physical or emotional signature of the user. If the updated physical or emotional signature shows improvement, then the particular product recommendations that the user selected for purchase may be understood as being beneficial for any other users having similar physical or emotional signatures, as the case may be.

The server 10 may determine that a physical signature shows improvement if, for example, the physical characteristics in the physical signature of the user have beneficially changed, for instance if the resting heart rate of the user decreases. On the other hand, a physical signature may show deterioration if, for example, the physical characteristics in the physical signature of the user have negatively changed, for instance the blood pressure of the user increases.

The server 10 may determine that an emotional signature shows improvement if, for example, the levels of cognitive-affective competencies in the emotional signature of the user have beneficially increased, for instance if the mood state of the user is repeatedly assessed to be positive. On the other hand, an emotional signature may show deterioration if, for example, the levels of cognitive-affective competencies comprised in the emotional signature of the user have negatively decreased, for instance if the mood state of the user is repeatedly assessed to be negative.

A deterioration in the physical or emotional signature of a user may be indicative that the recommendations carried out by the user are not effectively improving the user's overall wellbeing, and that alternative recommendations may be required. In such cases, server 10 can adjust the recommendations that are generated in response to determining the updated physical or emotional signature of the user and determining that the updated physical or emotional signature has deteriorated relative to the last known physical or emotional signature of the user.

Particular physical or emotional signatures may therefore be associated with particular personalized products or recommendations that have been shown to improve those physical or emotional signatures over time. Such associations, or data indicative of such associations, may be stored for example in databases 12 for future use, and may be accessed by servers 10 when determining the recommendations to generate for a user. Accordingly, when a new physical or emotional signature for the user session or a new user session is established for a user of product system 100, servers 10 may access databases 12 to identify a product recommendation or recommendations that have been shown to result in an improvement to similar physical or emotional signatures for other users of the system 100.

As an example, a user Jane decides to use product system 100 to determine her emotional signature by providing user data to the server 10 via the user device 16. Based on the information provided by Jane to her user device 16, and based on an analysis of the user data, including user data representing Jane's facial expressions, body language, tone of voice, measured biometrics, and behavioural patterns (based on text input provided by Jane in response to questions posed by the emotional wellbeing application), system 100 determines that Jane's emotional signature is similar to the emotional signature of Alice (another user). The system 100 recently (e.g. in a previous user session or as part of the same user session) recommended a product to Alice that will support her spending more time in the outdoors (e.g. recommended product involved nature), as Alice's emotional signature indicated a positive correlation between her mood and how much of her time was spent in nature outside. Over time, by repeatedly interfacing with the server 10 to provide updated user data, Alice's emotional signature as computed by the server 10 showed improvement as a result of spending more time in the outdoors. The server 10 therefore makes the same product recommendation to Jane, given his similar emotional signature to the emotional signature of Alice.

As another example, a user Paul decides to use product system 100 to determine his physical signature by providing user data to the server 10 via the user device 16. Based on the information provided by Paul including data representing Paul's physical activity, measured biometrics, fitness goals and behavioural patterns (based on text input provided by Paul in response to questions on a nutrition app), system 100 determine that's Paul's physical signature is similar to the physical signature of Andy (another user). The system 100 recently recommended a product to Andy that will support him eating more leafy vegetables, as Andy's physical signature indicated a positive correlation between progress towards his fitness goals and how many leafy vegetables he ate. Over time, by repeatedly interfacing with the server 10 to provide updated user data, Andy's physical signature as computed by the server 10 showed improvement as a result of eating more leafy vegetables. The server 10 therefore makes the same product recommendation to Paul, given his similar emotional signature to the emotional signature of Andy.

By generating and monitoring a physical or emotional signature for each user, the system 100 is able to build a dataset of physical or emotional signatures (stored as physical signature records or emotional signature records) and corresponding products or recommendations that are likely to improve individual physical or emotional signatures.

Additionally, the system 100 may enable individual users with similar physical and/or emotional signatures to be put in contact with one another, for example by providing access to relevant contact information. According to some embodiments, the system 100 may be used by team leaders, for example managers, in forming suitable teams. For instance, system 100 may be used to identify individuals that have similar emotional signatures and that may therefore work more efficiently or collaborate better when placed in the same team, or the system 100 may be used to identify individuals that have similar physical signature and that would likely be well suited to being each others' workout partners The system 100 can establish a communication session between multiple user devices 16, for example.

According to some embodiments, the system 100 may be configured to match people based on their physical and/or emotional signature, such that the matching persons can develop deep and meaningful romantic or friendship relationship, or the system 100 may be used to match a person with a coach or to a matching content. The system 100 may be used to identify individuals that have similar emotional signatures and therefore may connect on a deep and meaningful way. For example, based on users' input data (facial analysis, voice analysis, body analysis, textual input, activity input, biometrics input, etc.), as well as user's levels of cognitive-affective competencies and the individual's personality type, the system 100 can identify connections that may turn into a multi-year relationship or recommend activities that involve a compatible community or coaches that have high probability of long lasting connections between users and improved wellbeing.

The server 10 generates personalized products or product recommendations by matching users to certain (recommended) products to improve user's wellbeing. The server 10 aggregates and processes user data across multiple channels to extract metrics for determining a physical or emotional signature to provide improved product recommendations and trigger effects for a user's environment by actuating sensory actuators to impact the sensory environment for the user. The server 10 connects to interface 32 to display recommendations derived based on user data, activity metrics, and a physical or emotional signature of a user computed by the hardware processor accessing memory storing the user data and extracted metrics. The server 10 receives user data from multiple channels, such as different hardware devices, digital communities, events, live streams, and so on. The server 10 has hardware processors that can implement different data processing operations to extract activity metrics, cognitive-affective competency metrics, and social metrics by processing the user data from different channels.

The server 10 receives user data and, according to the method described herein before, processes the user data to determine the physical or emotional signature of such user. The server 10 can exchange data and receive output data used to generate the personalized products or recommendations, or determine the physical or emotional signature for use to generating the personalized products.

The server 10 can receive input data from different data sources or channels, such as different content providers (i.e., coaches, counsellors, influencers). The server 10 can aggregate and store content into a content center. As new input data is collected over an updated time period, the server 10 can re-compute updated physical and/or emotional signatures. Based on user's physical and/or emotional signature, the server 10 may recommend, for example, a product to help to improve user's wellbeing and/or achieve his/her goals. During usage of the product, the server 10 can receive data indicating user's performance from a data stream from an immersive hardware device (channels), such as for example, a smart watch, a smart phone, a smart mirror, or any other smart exercise machine (e.g., connected stationary bike) as well as any other sensors, such as sensors 24-26. Based on the collected data and the user's physical and/or emotional signature, the server 10 can dynamically adapt the personalized products or product recommendations. In one implementation, the recommendations generated by server 10 may take the form of a program to guide or shape matching pair/community interactions or experience. For example, the program may comprise one or more phases (daily, weekly, monthly, yearly programming). A program can be a series of activities that can map to time segments or intervals during the time period of the user session. Different activities and sessions may be recommended based on the phase. The server 10 can map activity data to phases. The intensity and volume of the sessions and products recommended may be varied linearly or non-linearly. Over time, through repeated interaction of the users with the user device 16, updated user data is captured and transmitted to the server 10 and the physical and emotional signatures of each user may be tracked or monitored based on the updated user data collected over time. The server 10 may change the recommendations based on the current physical and emotional signatures of the matching persons to maintain deep meaningful connections between the matched users. The server 10 can compute updated physical and emotional signatures at different intervals over the time period of a user session.

The physical and emotional signatures can be data structures of values (stored as records in non-transitory memory accessible by a hardware processor) that the system 1000 can compare to other data structures of values representing other physical or emotional signatures using different similarity measures or functions, for example. Different similarity measures can be used to identify similar physical or emotional signatures.

In some implementations, the methods and systems described herein can use user's physical or emotional signatures to make personalized products or product recommendations. Groups of users can increase social bonding through shared product experience. Therefore, the server 10 may be used to identify individuals that have similar physical or emotional signatures and to connect them by recommending the same or similar products. The server 10 can also generate social metrics for the user to make personalized products or recommendations.

In some implementations, the server 10 may manipulate external sensory environment (such as sound, lighting, smell, temperature, air flow in a room) to alter an individual's (or group of individuals) interoceptive ability to deliver greater physiological and psychological benefits during the product experience. The server 10 can manipulate the external sensory environment based on the activity inputs (e.g., type of activity, content, class intensity, class durations) received at user device 16, biometric inputs of users measured in real time during the class using the user device 16, as well as users' individual physical or emotional signatures calculated by the server 10 during previous sessions. For example, based on the emotional signature of the user or group of users, the server 10 may recommend a product to such user or group of users and then the product can be altered to match characteristics of the user or group of users. Depending on the recommended product and the emotional signature of the user or group of users, the server 10 can dynamically change the external sensory environment during the duration of the activity or experience to match the sequence/intensity of the activity/experience as well as users biometrics, or visual or audio cues/inputs.

The server 10 can use different data processing techniques to generate the physical and emotional signatures. For example, the server 10 can receive data sets (e.g. that can be extracted from aggregated data sources), extract metrics from the aggregated data sources, and generate the physical and emotional signatures for improved wellbeing using the extracted insights. The server 10 can transmit the physical and emotional signatures to other components of system 100. An interface 32 can connect to the server 10 to display visual effects based on the physical and emotional signatures. An interface 32 can connect to the server 10 to display the generated product or recommendation, or trigger updates to the interface based on the recommendation.

The server 10 monitors one or more users over a user session using one or more sensors. In some embodiments, the server 10 connects with interface 32 providing product recommendations for the user session. The server 10 has non-transitory memory storing product records, physical signature records, emotional signature records, and user records storing user data received from a plurality of channels, for example.

The user data can involve a range of data captured during a time period of the user session (which can be combined with data from different user sessions and with data for different users). The user data can be image data relating to the user, text input relating to the user, data defining physical or behavioural characteristics of the user, and audio data relating to the user.

The user device 16 can be programmed with executable instructions for user interface for obtaining user data for a user session over a time period. The user device 16 transmits a product request for the user session to server 10, and updates its interface for providing product recommendations for the user session received in response to the product request.

The server 10 can be coupled to non-transitory memory to access the product records, the physical signature records, the emotional signature records, and the user records.

The server 10 is programmed with executable instructions to transmit the product recommendations to the interface 32 over a network in response to receiving the product request. The server 10 is programmed with executable instructions to compute the product recommendations based on metrics extracted from the received user data in this example embodiment. The server 10 can compute activity metrics, cognitive-affective competency metrics, and social metrics using the user data for the user session and the user records. The server 10 can extract metrics from the user data to represent physical metrics of the user and cognitive metrics of the user. The server 10 can use both physical metrics of the user and cognitive metrics of the user to determine the physical or emotional signature for the user during the time period of the user session. The server 10 can compute multiple physical and emotional signatures for the user at time intervals during the time period of the user session. The server 10 computes multiple physical and/or emotional signatures which can trigger computation of updated product recommendations and updates to the interface. The physical and emotional signatures use both physical metrics of the user and cognitive metrics of the user during the time period of the user session.

The server 10 can transmit the computed physical and emotional signatures to the interface 32 in response to the request, for example. The server 10 can use user data captured during the user session and can also use user data captured during previous user sessions or user data for different users. The server 10 can aggregated data from multiple channels to compute the product recommendations to trigger updates to the interface 32, or an interface on a separate immersive hardware device in some examples.

The server 10 can process different types of data by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis.

The server 10 can compute one or more states of one or more physical characteristics or cognitive-affective competencies of the user based on the physical metrics, the cognitive-affective competency metrics and the social metrics. The server 10 can compute a physical or emotional signature of the user based on the one or more states of the one or more physical characteristics, cognitive-affective competencies of the user and using the physical signature records or emotional signature records. The server 10 can compute the product recommendations based on the physical signature of the user, emotional signature of the user, the activity metrics, the product records, and the user records.

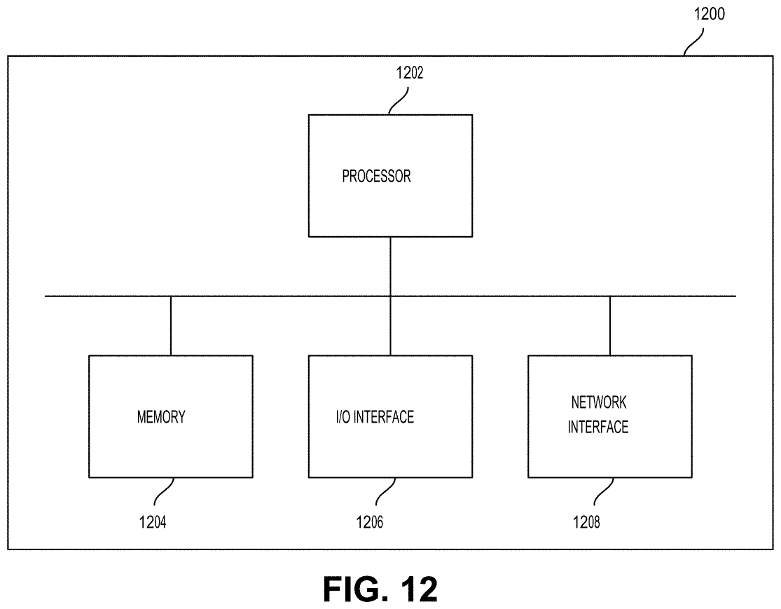

FIG. 12 shows an example schematic diagram of a computing device 1200 that can implement aspects of embodiments, such as aspects or components of user device 16, servers 10, databases 12, system 100, or interface 32. As depicted, the device 1200 includes at least one hardware processor 1202, non-transitory memory 1204, and at least one I/O interface 1206, and at least one network interface 1208 for exchanging data. The I/O interface 1206, and at least one network interface 1208 may include transmitters, receivers, and other hardware for data communication. The I/O interface 1206 can capture user data for transmission to another device via network interface 1208, for example.

The server 10 monitors one or more users over a user session using user device 16 with sensors. In some embodiments, the interface 32 displays product recommendations for the user session. The server 10 has non-transitory memory storing product records, physical signature records, emotional signature records, and user records storing user data received from a plurality of channels, for example.

The user data can involve a range of data captured during a time period of the user session (which can be combined with data from different user sessions and with data for different users). The user data can be image data relating to the user, text input relating to the user, data defining physical or behavioural characteristics of the user, and audio data relating to the user.

The interface 32 resides on a hardware processor (which can be at user device 16 or a separate computing device) programmed with executable instructions for obtaining user data for a user session over a time period. The interface 32 transmits a product request for the user session to the server 10, and updates with personalized products or product recommendations for the user session received in response to the product request.

The server 10 is programmed with executable instructions to transmit the personalized products or product recommendations to the interface 32 over a network in response to receiving the product request from the interface 32. The server 10 is programmed with executable instructions to compute the product recommendations by: computing activity metrics, cognitive-affective competency metrics, and social metrics using the user data for the user session and the user records. The server 10 can extract metrics from the user data to represent physical metrics of the user and cognitive metrics of the user. The server 10 can use both physical metrics of the user and cognitive metrics of the user to determine the physical and/or emotional signature for the user during the time period of the user session. The server 10 can compute multiple physical and/or emotional signatures for the user at time intervals during the time period of the user session. The server 10 computes multiple physical and or emotional signatures which can trigger computation of updated product recommendations and updates to the interface 32. The physical and emotional signatures use both physical metrics of the user and cognitive metrics of the user during the time period of the user session.

The server 10 can use user data captured during the user session and can also use user data captured during previous user sessions or user data for different users. The server 10 can aggregated data from multiple channels to compute the personalized products or product recommendations to trigger updates to the interface.

The server 10 can process different types of data by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis. The data can be used for generating the personalized products.

The server 10 can compute one or more states of one or more physical characteristics of the user based on the physical metrics. The server 10 can compute a physical signature of the user based on the one or more states of the one or more physical characteristics of the user and using the physical signature records.

The server 10 can compute one or more states of one or more cognitive-affective competencies of the user based on the cognitive-affective competency metrics and the social metrics. The server 10 can compute an emotional signature of the user based on the one or more states of the one or more cognitive-affective competencies of the user and using the emotional signature records.

The server 10 can compute the personalized products or product recommendations based on the physical signature of the user, the emotional signature of the user, measurement and movement metrics, the product records, and the user records. The system 100 has a user device 16 comprising one or more sensors for capturing user data during the time period, and a transmitter for transmitting the captured user data to the server 10 over the network to compute the personalized products or product recommendations.

The interface 32 receives a product request, transmits the request to the server 10, and updates to provide a personalized product or product recommendation in response to the request. The interface 32 is a tool to provide the visualizations of products derived based on user data, activity metrics, and a physical or emotional signature of a user.

The product request can relate to a time period and a product recommendation generated in response to the request can relate to the same time period. In some embodiments, the server 10 can determine the product recommendation. The interface 32 can display the product recommendation or otherwise provide the product recommendation such as by audio or video data. The interface 32 is shown on a computing device with a hardware processor in this example.

In some embodiments, the interface 32 can transmit the product request to the server 10 to determine a recommendation or generate personalized product. The interface 32 can transmit additional data relating to the product request such as a time period, user identifier, application identifier, or captured user data to the server 10 to receive an product recommendation in response to the request.

The server 10 can process the user data to determine the physical or emotional signature of such user, or the server 10 can communicate with other components of the system 100 to compute the physical or emotional signature. The sever 10 can use the physical or emotional signature for the user for the time period to generate the recommendation for the interface 32.

For example, in some embodiments, the interface 32 can determine a physical or emotional signature of the user for the time period, and send the physical or emotional signature for the time period to the server 10 along with the product request. The interface 32 can store instructions in memory to determine the physical or emotional signature for a user for a time period. The interface 32 is shown on a computing device with non-transitory memory and a hardware processor executing instructions to obtain user data and display product recommendations. For example, the interface 32 can obtain user data by connecting to a user device 16 along with the sensors 24-28 collecting the user data for a time period. The interface 32 can connect to the separate hardware server 10 to exchange data and receive output data used to generate the recommendations or determine the physical or emotional signature.

The interface 32 can obtain user data from the multiple channels 1040, or collect user data from user device 16 (with sensors) for computing the physical or emotional signature. In other embodiments, the server 10 determines the physical or emotional signature of the user for the time period in response to receiving the product request from the interface 32. Using the server 10 to compute the physical or emotional signature for the user for the time period can offload the computation of the physical or emotional signature for the user for the time period (and required processing resources) to the server 10 which might have greater processing resources than the interface 32, for example. The server 10 can have secure communication paths to different sources to aggregated captured user data from different sources, to offload data aggregation operations to the server 10 which might have greater processing resources than the interface 32, for example.

In some embodiments, the interface 32 can capture user data (via I/O hardware or sensors of computing device) for use in determining the physical or emotional signature of the user for the time period and for measurement data or movement data. In some embodiments, one or more user devices 16 capture user data for use in determining the personalized product or product recommendation. In some embodiments, the interface 32 can reside on the user device 16, or the interface 32 can reside on a separate computing device than the user device 16.

In some embodiments, the interface 32 can transmit the captured user data to the server 10 as part of the product request, or in relation thereto. In some embodiments, the interface 32 extracts measurement metrics, movement or activity metrics, physical metrics, cognitive-affective competency metrics, and social metrics by processing captured user data. The captured user data can be distributed across different devices and components of the system 100. The server 10 can receive and aggregate captured user data from multiple sources, including channels, content centre, user device 16, and server 10. In some embodiments, the interface 32 can extract activity metrics, physical metrics, cognitive-affective competency metrics, and social metrics by processing user data from multiple sources, and provide the extracted metrics to the server 10 to compute the physical or emotional signature and personalized products or product recommendations.

In some embodiments, in response to receiving the request from the interface 32, the server 10 can extract activity metrics, physical metrics, cognitive-affective competency metrics, and social metrics by processing captured user data for the time period. The server 10 can register different interfaces 32 to link an application identifier to a user identifier. The server 10 can extract an application identifier from the request in some embodiments, to locate a user identifier to retrieve relevant records.

The server 10 can receive and aggregate captured user data from multiple sources, including channels, content centre, user device 16, and applications. In response to receiving the request from the interface 32, the server 10 can request additional captured user data relevant to the time period from different sources. The server 10 can use the aggregated user data from the multiple sources to extract activity metrics, physical metrics, cognitive-affective competency metrics, and social metrics by processing the captured user data for the time period. The user data from the multiple sources can be indexed by an identifier (e.g. user identification) so that the server 10 can identify user data relevant to a specific user across different data sets, for example. The server 10 has hardware processors that can implement different data processing operations to extract activity metrics, physical metrics, cognitive-affective competency metrics, and social metrics by processing the user data from different channels, content centre, user device 16, interface 32. The server 10 has a database or user records, physical signature records, emotional signature records, and product records. The user records can store extracted activity metrics, physical metrics, cognitive-affective competency metrics, and social metrics for a user across different time periods, for example. The user records can store product recommendations for a user for different time periods based on the extracted activity metrics, physical metrics, cognitive-affective competency metrics, and social metrics for the different time periods, for example.

The server 10 uses the extracted activity metrics, physical metrics, cognitive-affective competency metrics, and social metrics to determine the personalized product or product recommendation for the time period. The server 10 can extract measurement metrics, activity metrics, physical metrics, cognitive-affective competency metrics, and social metrics, or can receive extracted activity metrics, physical metrics, cognitive-affective competency metrics, and social metrics from the interface 32 (or different channels 1040, content centre 1020, user device 16), for example, or a combination thereof. The server 10 can aggregate extracted product metrics, physical metrics, cognitive-affective competency metrics, and social metrics for the user for the time period to determine the physical or emotional signature of the user and the product recommendation.

In some embodiments, the server 10 aggregates user data from multiple sources (channels 1040, user device 16, content centre 1020) to leverage distributed computing devices so that the interface 32 does not have to collect all the user data from all the different sources. The channels, user device 16, content centre can have different hardware components to enable collection of different types of data. In some embodiments, the system 100 distributes the collection of user data across these different sources to efficiently collect different types of data from different sources. The server 10 can have secure communication paths to different sources to aggregated captured user data from different sources in a secure way at a central repository, for example. Captured user data from multiple sources may contain sensitive data and the server 10 can provide secure data storage. This can alleviate the need for the captured user data from multiple sources (with sensitive data) to be stored locally on different devices, which might create security issues, for example. This can offload data aggregation operations to the server 10 which might have greater processing resources than the interface 32, for example.

In some embodiments, the server 10 computes the physical or emotional signature for the user for the time period. The interface 32 exchanges data with the server 10 for computing the physical or emotional signature. As noted, the server 10 can send requests for updated user data, receive updated user data in response from multiple channels, and aggregate the user data from the multiple channels, such as different hardware devices, digital communities, events, live streams, and so on, for computing the physical or emotional signature. The server 10 can store the aggregated user data in user records, for example.

As new input data is collected by the server 10 (or interface 32, channels, user device 16, content centre) over an updated time period, server 10 can compute a physical or emotional signature for the user for the updated time period. If a new product request is received by the server 10 for an updated time period, the server 10 can compute a physical or emotional signature for the user for the updated time period. The physical or emotional signature for the initial time period can be different from the physical or emotional signature for the updated time period. The physical or emotional signature for the updated time period is used to determine the product recommendations. Accordingly, an updated physical or emotional signature for the updated time period can trigger different product recommendations than the product recommendations determined based on the physical or emotional signature for the previous time period.

In some embodiments, interface 32 sends a request to the server 10 to compute the physical or emotional signature for the updated time period. In response, the server 10 can compute a new physical or emotional signature for the updated time period and can also determine new product recommendations based on the emotional signature for the updated time period. In response, the server 10 can send data for the physical or emotional signature for the updated time period to the interface 32, and can also send the new product recommendations based on the physical or emotional signature for the updated time period. Using the server 10 for computation can offload processing requirements to separate hardware processors of the server 10.

The server 10 stores data for the physical and emotional signatures in a database of physical signature records and emotional signature records. Each physical signature record and emotional signature record can be indexed by a user identifier, for example. Each physical signature record and emotional signature record can indicate the time period, a value corresponding to the computed physical or emotional signature for the time period, and extracted metrics, for example. The physical signature record and emotional signature records can also store any product recommendations for the time period. The physical and emotional signature records can include historic data about previous physical and emotional signature determinations for the user for different time periods. The physical and emotional signature records can include historic data about previous physical and emotional signature determinations for all users of the system. The historic data for physical and emotional signature records can include time data corresponding to time periods of user data used to compute physical and emotional signatures. Accordingly, server 10 can compute a physical or emotional signature for a user for a time period and store the computed physical of emotional signature in the database of physical and emotional signature records with a user identifier, values for the computed physical or emotional signature, and the time period. The physical or emotional signature can be a data structure of values. The server 10 can define parameters for the data structure of values that can be used to compute values for a physical or emotional signature based on the captured user data for the time period. The server 10 can compare to other data structures of values representing other physical or emotional signatures using different similarity measures, for example. Different similarity measures can be used to identify similar physical or emotional signatures. The server 10 maps physical and emotional signatures (data structure of values) to user records and product records.

In some embodiments, the server 10 has a database of user records with user identifiers and user data. Each user record can be indexed by a user identifier, for example. The server 10 can identify a set of physical and emotional signature records based on a user identifier, for example, to identify physical or emotional signatures determined for a specific user or to compare physical or emotional signatures for a specific user over different time periods.

The server 10 stores data for the product recommendations in a database of product records. Each product recommendation can be indexed by a product identifier, for example. The product records can define different products, parameters or variables for the products, identifiers for the products, and other data. The product records can include historic data about previous product recommendations for the user, and previous product recommendations for all users of the system 100. The historic data for product records can include time data that can map to time periods of physical or emotional signatures. A user record and/or a physical or emotional signature record can also indicate an activity identifier to connect the user record and/or the physical or emotional signature to a specific product record. For example, the server 10 can compute a physical or emotional signature for a user for a time period based on user data, and determine an product recommendation for the user for the time period. The product recommendation can correspond to an product recommendation record indexed by a product identifier. The user record can store the product identifier and the time period to connect the user record to a specific product record. The physical signature record or emotional signature record might also indicate the user identifier, or an emotional signature identifier. The user record can also indicate the physical or emotional signature identifier to connect the user record, the specific product record, and the physical or emotional signature record. A physical or emotional signature record might also indicate parameters for computing different types of physical or emotional signatures using different types of data. The physical or emotional signature record might also have a model for computing a physical or emotional signature for a time period. The physical or emotional signature record might also indicate different product identifiers to connect a physical emotional signature to an product recommendation record.

Based on user's physical or emotional signature, the server 10 may transmit data to the application to update the interface 32. The data can be instructions for displaying visualizations of the product (or recommended product) on the interface 32 or for generating audio or video data at the interface 32, for example.

The server 10 and the interface 32 can connect using an application programming interface (API) and exchange commands (including the product request) and data using the API. The interface 32 can receive instructions from the server 10 to provide product recommendations. For example, the interface 32 can provide a virtual coach interface that provides product recommendations over time periods to help improve the user's wellbeing and/or achieve his/her goals. The interface 32 can exchange commands and data with the server 10 using the API to receive product recommendations and automatically update the virtual coach interface to automatically provide the product recommendations. The interface 32 can use the virtual coach interface to prompt for user data, and can transmit collected user data to the server 10 using the API.

The interface 32 can automatically update displayed visualizations to provide a product recommendation for the time period. The interface 32 can continue to monitor the user (via collection of user data) during usage of the product to collect feedback data, which can be referred to as user data. The interface 32 can receive positive or negative feedback about the product recommendation for the time period. For example, the interface 32 updates to provide a first product recommendation for the time period and receives negative feedback about the first product recommendation for the time period. The interface 32 can exchange commands and data with the server 10 using the API to receive a second product recommendation for the time period and communicate the negative feedback. The server 10 can store the negative feedback in a user record with an activity identifier for the first product recommendation for the time period, for example, or otherwise store the negative feedback in association with the first product recommendation.

During performance of the activities, the system 100 can receive data indicating user's performance from a data stream from a different channels such as immersive hardware devices (as an example user device 16), such as for example, a smart watch, a smart phone, a smart mirror, or any other smart exercise machine (e.g., connected stationary bike) as well as any other sensors, such as sensors 24-26. For example, the user device 16 can be a smart mirror with a camera and sensors to capture user data. The user device 16 that is a smart mirror can also have the interface 32, for example, to provide product recommendations to the user for the time period. The application 1010 can send the product request along with the captured user data from the user device 16 (smart mirror) to the server 10 using the API to receive an product recommendation for the time period to update the interface. Accordingly, the interface 32 can provide product recommendations for different time periods and can also have sensors to capture user data for the time periods.

Based on the collected data and user's physical or emotional signature, the server 10 can dynamically adapt by providing updated product recommendations over different time periods, or updated product recommendations for the same time period based on feedback from the interface for previous product recommendations. In one implementation, the recommendations generated by server 10 may take the form of a program of multiple product recommendations for a time period (or time segments) to guide or shape matching pair/community interactions or experience. For example, the program may comprise one or more phases of product recommendations for different time periods (daily, weekly, monthly, yearly programming). The server 10 can compute different product recommendations and sessions based on the phase and current time period. Over time, through repeated interaction of the users with the emotional interface 32 on their user device 16, updated user data is captured by the interface 32 and sent to the server 10 for tracking and storage. Over time, the server 10 can track and monitor the physical or emotional signature of each user based on the updated user data collected over time. The server 10 may define a program as a set of product recommendations. The server 10 may change the set of product recommendations. The server 10 may change the products based on the current physical or emotional signatures of the matching persons to align the set of product recommendations to help maintain deep meaningful connections between the matched users.

The server 10 can use physical or emotional signatures to make product recommendations for a group of users. The server 10 can generate the same product recommendation for each user of the group, for example, based on the physical or emotional signatures computed for each user of the group. Group exercises improve individual wellbeing and increase social bonding through shared emotions and movement. Therefore, the server 10 may be used to identify individuals that have similar physical or emotional signatures and connect them by generating the same product recommendation related to their group exercise (or other group activity) for a set of identified users or peers. Each user in the group can be linked to a interface 32 and the server 10 can send the same product recommendation to each of the interface 32 for the set of identified users and continue to monitor the user data for a set of identified users by capturing additional user data after providing the same product recommendation. The server 10 can also generate social metrics for the user to make recommendations for the set of identified users.

In some implementations, the server 10 may manipulate external sensory environment by controlling connected sensory actuators (such as sound, lighting, smell, temperature, air flow in a room) as part of the product experience. The sensory actuators can be part of a building automation system, for example, to control components of the building system to coordinate with content delivered to interface 32 or user device 16. The server 10 can transmit control commands to sensory actuators as part of the process of generating product recommendations, computing physical or emotional signatures, or ongoing monitoring of users by capturing additional user data.

The server 10 may control connected sensory actuators to alter a user's (or group of users) interoceptive ability to deliver greater physiological and psychological benefits during the class/experience. The server 10 can manipulate the connected sensory actuators based on the product recommendations (e.g., type of activity, content, class intensity, class durations), feedback received at user device 16 or interface 32, biometric inputs of users measured in real time during the class using the user device 16, as well as users' individual physical or emotional signatures calculated by the system 100 during previous sessions.

For example, based on the physical or emotional signature of the user or group of users, the server 10 may generate an product recommendation for such user or group of users and then sound tempo or volume related to the product recommendation can be altered to match the recommendation by the server 10 controlling sensory actuators. Depending on the recommended product and the physical or emotional signature of the user or group of users, the server 10 can dynamically change the external sensory environment during the duration of the activity or experience to match the sequence/intensity of the activity/experience as well as users biometrics, or visual or audio cues/inputs.

The interface 32 or server 10 can use different data processing techniques to generate the physical or emotional signature. For example, the interface 32 or the server 10 can receive data sets (e.g. that can be extracted from aggregated data sources), extract metrics from the aggregated data sources, and generate the physical or emotional signature for improved wellbeing using the extracted metrics.

In some embodiments, the interface 32 can transmit the physical or emotional signature to the server 10 along with the product request. In response, the interface 32 updates its interface to display visual effects based on the physical or emotional signature, and also based on the product recommendation received by the server 10. The interface 32 can connect to the server 10 to display the generated recommendation at the interface, or trigger other updates to the interface based on the recommendation (e.g. change visualizations provided by the interface 32).

While in the above-described embodiment the processing of the user data, the determination of the physical or emotional signatures, and the generation of the personalized products or recommendations have been described as being performed by hardware servers 10, in other embodiments such steps may be performed by user device 16, provided that user device 16 has access to the required instructions, techniques, and processing power. Servers 10 can have access to greater processing power and resources than user devices 16, and therefore may be better suited to carrying out the relatively resource-intensive processing of user data obtained by user devices 16 and across channels.

In some embodiments, the server 10 stores classifiers for generating data defining physical or behavioural characteristics of the user. The server 10 can compute the activity metrics, physical metrics, cognitive-affective competency metrics, and social metrics using the classifiers and features extracted from multimodal feature extraction. The multimodal feature extraction can extract features from image data, video data, text data, and so on. The classifiers can be models to generate output data that corresponds to different activity metrics, physical metrics, cognitive-affective competency metrics, and social metrics. The classifiers can be trained on user data and can be updated based on feedback data.

In some embodiments, the server 10 stores user models corresponding to the users. The server 10 can retrieve a user model corresponding to a user and computes the physical or emotional signature of the user using the user model. The server 10 can compute the recommendations or personalization of products based on the physical or emotional signature of the user using the user model.

In some embodiments, the user device 16 connects to or integrates with an immersive hardware device that captures the audio data, the image data and the data defining the physical or behavioural characteristics of the user. The user device 16 can transmit the captured data to the server 10 for processing to compute the recommendations or personalization of products. The user device 16 connects to the immersive hardware device using Bluetooth, or other communication protocol.

In some embodiments, the server 10 stores a content repository and has a content curation engine that maps the product recommendations to recommended content and transmits the recommended content to the interface 32.

In some embodiments, the interface 32 further comprises a voice interface for communicating product recommendations for the user session received in response to the product request. The voice interface can use speech/text processing, natural language understanding and natural language generation to communicate product recommendations and capture user data. The interface 32 can implement speech-to-text translation and process text data on the specific word usage and variance using natural language processing.

In some embodiments, the interface 32 can access memory storing mood classifiers to capture the data defining physical or behavioural characteristics of the user.

In some embodiments, the server 10 computes activity metrics, cognitive-affective competency metrics, and social metrics with classifiers using the user data for the user session and the user records and multimodal feature extraction that processes data from multiple modalities. The server 10 uses multimodal feature extraction for extracting features and correlations across the image data, the data defining the physical or behavioural characteristics of the user, the audio data, and the text input. Multimodal signal processing analyzes user data through several types of measures, or modalities such as facial analysis; body analysis; eye tracking; behavioural analysis; social network or graph analysis; location analysis; user activity analysis; voice analysis; and text analysis, for example, and extracts features from the processed data.

In some embodiments, non-transitory memory stores classifiers for generating data defining physical or behavioural characteristics of the user, and the server 10 computes the activity metrics, physical metrics, cognitive-affective competency metrics, and social metrics using the classifiers and features extracted from the multimodal feature extraction.

The interface 32 has an interface that receives a product request and provides an product recommendation or personalized product in response to the request. The interface 32 provides the recommendations derived based on user data, activity metrics, and a physical or emotional signature of a user. The interface 32 can provide product recommendations for different user sessions that can be defined by time periods. The server 10 can process user data based on the different user sessions defined by time periods. For example, interface 32 can send a product request to the server 10 to start a user session for a time period. The user session maps to a user by a user identifier. The user session can define a set of captured user data (including captured real-time data), one or more physical or emotional signatures, and one or more product recommendations. A user session link a group of users in some examples. Each user session can have a product request and a corresponding one or more product recommendations. Each user session can be identified by the system 100 using a session identifier stored in records of database 12. The product request can indicate the session identifier, or the server 10 can generate and assign as session identifier in response to receiving a product request. The server 10 and the interface 32 can exchange the session identifier via the API, for example. The server 10 can store extracted metrics in association with a session identifier to map the data values to user sessions. The server 10 can use data values from previous user sessions to compute physical or emotional signatures and product recommendations for a new user session. The previous user sessions can relate to the same user or different users.

The interface 32 can provide product recommendations or personalized products for the user sessions. The user devices 16 can also have sensors to capture (near) real-time user data during the time period of the user session (or proximate thereto) to determine the physical or emotional signature of a user for the time period. A user session can be defined by one or more time periods or segments of a time period. A user session can map to one user identifier or multiple user identifiers.

The server 10 or hardware server 10 receives input data from different data sources, such as content center 1020, user devices 16, and channels 1040 to compute different metrics for computation of the physical and emotional signatures. The server 10 or hardware server 10 computes the physical or emotional signature for the user for the time period of the user session using the captured (near) real-time user data, along with other user data. The server 10 can access records in databases 12, for example. The server 10 can compute similarity measures across records for computation of the physical or emotional signature of the user for the time period of the user session.

The product request can relate to a time period of the user session and the product recommendation or personalized product generated in response to the request can relate to the same time period. The system 1000 can store the product with the session identifier in records. In some embodiments, the interface 32 can determine the product or the physical or emotional signature. In some embodiments, the interface 32 can extract metrics from captured user data and transmit the extracted metrics to the server 10. The interface 32 can display the product recommendation or otherwise provide the product recommendation such as by audio or video data.

As an example illustration, there can be multiple users devices 16 with sensors. The user devices 16 can connect to server 10 to exchange data for user sessions. The server 10 or hardware server 10 can aggregate or pool data from the multiple users devices 16 and send product recommendations to interfaces 32. The server 10 can coordinate timing of the real-time data collection from a group of users corresponding to a set of user devices 16 and can coordinate timing and content of product recommendations for the interfaces 32 for each user of the group of users. A group of users can be assigned to a user session, for example, to coordinate data and messages. For example, server 10 can generate the same product recommendation for transmission to interface 32 for each user of the group of users of the user session. The interface 32 can be linked to a user by a user identifier that can be provided as credentials at the interface or generated using data retrieved by the user device 16. The user identifier can map to a user record in the database 12. The session identifier can also map to one or more user identifiers in the database 12. During a registration process, the interface 32 can exchange the user identifier with the server 10 or hardware server 10 via the API, for example.

The example can involve server 10 exchanging data between multiple interfaces 32 and multiple user devices 16 with sensors. The server 10 or hardware server 10 can have increased computing power to efficiently compute data values from the aggregated user data. Each user device 16 does not have to store the aggregated user data and does not have to process similarity measures across a group of users. Each user device 16 does not have to exchange data with all the user devices 16 in order to access the benefits of data aggregation. Instead, the user device 16 can exchange data with the server 10. The server 10 can store the aggregated user data and process similarity measures across a group of users and exchange data with the user device 16 based on the results of its computations. The user device 16 can capture real-time user data during user sessions for the server 10 or hardware server 10, or can perform computations for the user session using the real-time user data and data received from the server 10. The user device 16 can extract metrics from captured user data and transmits the extracted metrics to the server 10. The user device 16 can exchange data and commands with the server 10 during user sessions using the API. The extracted metrics can correspond to parameters for the API, as an example. The user device 16 can transmit extracted metrics to the server 10 using the API. The user device 16 can extract metrics from captured user data so that the metrics might not reveal all sensitive user data. In some embodiments, the user device 16 can transmit the metrics to the server 10 using the API instead of all the sensitive user data.

The server 10 or hardware server 10 can serve a large number of user devices 16 and interfaces 32 to scale the system 100 to collect a corresponding large amount of data for the computations. The system 100 can have multiple hardware servers 10 to serve sets of user devices 16, for example, and provide increased processing power and data redundancy.

The server 10 can receive user data relating to a user for user sessions from a plurality of channels. The user data involves different types of data such as image data relating to the user, text input relating to the user, data defining physical or behavioural characteristics of the user, and audio data relating to the user. The server 10 can implement pre-processing steps on the raw data received from different channels. Examples include importing data libraries; data cleaning or checking for missing values/data; smoothing or removing noisy data and outliers; data integration; data transformation; and normalization and aggregation of data.

The interface 32 can exchange data and commands with the server 10 using the API, such as metrics extracted from the captured user data. The interface 32 or the server 10 can generate activity metrics, cognitive-affective competency metrics, and social metrics by processing the user data using one or more hardware processors configured to process the user data from the plurality of channels. This includes captured user data for a time period given that the product recommendation corresponds to the time period. The captured user data for the time period is used to compute a physical or emotional signature for the user for the time period.

The activity metrics, cognitive-affective competency metrics, and social metrics define “physical” metrics and “cognitive” metrics for the system 100. Raw data is ingested by the system 100 from the different channels and mapped to these definitions of “physical” metrics and “cognitive” metrics by the system 100. The metrics can have corresponding values based on the processed user data. The system 100 abstracts from the raw user data using the “physical” metrics and “cognitive” metrics to provide an improved way to compute values for physical and emotional signatures for the user for the time period.

For example, the system 100 measures the physiological condition of the user using sensors (accelerometer, heart rate monitor, breath rate monitor) to capture real-time user data and processes user data to measure physiological conditions (e.g. measuring heart rate, heart rate variability) by assigning values to different metrics. The system 100 can define a ‘physical’ metric or a fluidity score during a workout activity that can be computed by user data captured using physiological sensors of user device 16 with or without a camera, for example. The system 100 can define a connectedness metric using heart rate and heart rate variability during a workout activity that involves a product, as another example.

For example, the system 100 measures cognitive metrics using definitions based on text inputs (with predefined answers and free text answers with predefined features extracted to predefined questions), daily behaviour (e.g. extracted from user's device 16 like app usage, music consumption, number of outgoing calls), voice (power spectrum of the speech signal can correlate emotions like neutral, anger, joy, sadness), body language extracted from image data (posture, special location and orientation of joints like wrist and hands can correlate emotions like happy, sad, surprise, fear, anger, disgust, neutral); eye movement (saccade duration, fixation duration, pupil diameter can correlate to positive, neutral or negative emotional state). Another example is brain activity data (e.g. N400 response).

As another example, the system 100 can measure cognitive metrics using higher level state definitions such as intention/awareness, attention, motivation, emotion regulation, perspective-taking/insight, self-compassion and compassion towards others.

The system 100 can measure physical metrics and cognitive metrics from captured user data for a user session and then compute the physical or emotional signature for the user session using the physical metrics and cognitive metrics for generating the product recommendations or personalized products. The system 100 can measure physical metrics and cognitive metrics from text base interaction and free text response, and extracts features from the free responses for computing to the physical or emotional signature. A user can be in front of a mirror device with a camera to capture images of gestures and audio data of speech for the user session which can be used to compute additional metrics such as tone or body posture.

The system 100 can measure physical metrics as state metrics, such as being “happy” can be smile detected in image data or posture or tone from audio data. The system 1000 can measure trait metrics or personality more constant features. For example, to measure the level of attention or focus a user has at a given time the interface can prompt a predefined question: ‘how focused are you feeling right now?’ with a 1-7 Likert scale response. The interface 32 can ask a specific or general question and extract any features related to feeling focused through a free text response. The system 100 can also consider communication messages between users, such as text conversation data between two users and extract features related to a user describing feeling focused. The system 100 can also consider reaction times to digital interactions on the phone or other devices (e.g. button clicks). The system 100 can also consider device 16 usage data to measure how much time a user was on task or focused, or off-task and not focused in the day. The system 100 could use visual eye tracking to measure attention and focus to a particular task.

The interface 32 or server 10 can extract metrics from image data and the data defining the physical or behavioural characteristics of the user, using at least one of: facial analysis; body analysis; eye tracking; behavioural analysis; social network or graph analysis; location analysis; user activity analysis. Examples of different facial feature extraction techniques and image processing techniques include observation techniques such as those based on the Facial Action Coding System where observable activity of specific muscle group are labeled and coded as action units by human coders, record muscles activities with facial electromyography; facial expression coding system (FACES) developed by Berkeley (https://esilab.berkeley.edu/wp-content/uploads/2017/12/Kring-Sloan-2007.pdf) the entire contents of which is hereby incorporated by reference.

The interface 32 or server 10 can extract metrics from the audio data, using different voice processing techniques. For example, metrics can be values based on non-linguistic verbal interactions for emotion states (e.g. laughter, sighs)

The interface 32 or server 10 can extract metrics from the text input using text analysis and different natural language understanding techniques to extract features from text data, including meaning and sentiment analysis.

The interface 32 or server 10 can compute activity metrics, cognitive-affective competency metrics, and social metrics. The interface 32 or server 10 can determine, based on the cognitive-affective competency metrics and social metrics generated from the processed user data, one or more states of one or more cognitive-affective competencies of the user. Examples of state classification happy, sad, disgust, moment of insight, giving, compassion, compelled to help, jealousy, energized, being focus, surprise, fear, anger, curious, aware, unaware. The interface 32 or server 10 can define multiple states and select states for user sessions or time periods. The definitions for states can relate to ‘readiness to grow’ as another example.

The interface 32 or server 10 can compute a physical or emotional signature of the user for the user session based on the one or more states of the one or more cognitive-affective competencies of the user. The system 100 can map states to the physical or emotional signature values or parameters. By way of example for physical fitness, training over years contributes to a general fitness level which does not change quickly. If a user has done hard training recently, the day after the training session the user might be really tired and so their readiness to train might be low. The system 100 can consider metrics for a user computed based on data captured before or prior to the time period of the user session, along with metrics for the user computed based on data captured during the time period of the user session. The system 100 can use a weighting or ratio for the metrics to compute the physical or emotional signature or additional metrics for the session. The emotional signature can be computed using metrics for different dimensions of emotion, such as Awareness, Regulation, Compassion (ARC) dimensions of emotion. Within each dimension there are different states that could be detected by the system 100 that would be attributed to that dimension. For awareness, the system 100 can define subdimensions like reflectiveness, mindfulness and purposefulness. The user device 16 can display an initial questionnaire to receive input data for a user session to measure as a trait level metric. However, with different real time data inputs the system 100 can measure discrete states at different time intervals (using data corresponding to the different time intervals) over a time period or across different user sessions. For example, a user would be in a state of reflectiveness when they are labeling a current or past experience and expressing things like emotions or feelings they had during that experience in words either spoken or written. To detect this state, a person's spoken language or written language could be processed and features extracted that relate to the expression of emotions in relation to an event.

The interface 32 or server 10 can define physical or emotional signatures as functions or sets of variables or values. An emotional signature definition can model ARC dimensions and consider values for metrics for ARC dimensions as profiles of values (metric 1, metric 2, metric 3) with different versions or combinations depending on the values that can be assigned. An example is profile of values is (A, R, C) where each value can be high or low, with different versions of profiles such as: (high-high-high) (high-high-low) (high-low-high) (high-low-low) (low-low-high) (low-low-low) (low-high-high) (low-low-high). The different versions of profiles can map to different emotional signatures. The profiles can be stored in records of database 12, for example.

The interface 32 or server 10 can select a physical or emotional signature from a group of physical or emotional signatures using confidence scores or distribution rules, for example. As an example, the rules can correspond to default to the population distribution work or the profile that best represents most users, e.g. lower on self-compassion or higher on flexibility. The interface can also prompt for more information to capture additional user data (e.g. digital or virtual coach or conversational agent style interface) to select a physical or emotional signature from the group of physical or emotional signatures.

The server 10 can automatically generate, based on the physical or emotional signature of the user and the activity metrics, one or more product recommendations or personalized products for display at the interface 32. The server 10 transmits the product recommendations to the interface 32 in response to a product request. Recommendations can be based on thresholds of scores from predefined questions and Likert scale responses. Recommendations can be based on advanced data points and complex data collection and analysis methods.

The interface 32 can provide an automated coaching application to provide automated product recommendations or personalized products for user sessions using physical and cognitive metrics extracted from captured user data.

The interface 32 can be a mobile companion application (residing an a computer device) for a separate hardware device that captures user data. The separate hardware device can also have an interface that can deliver recommendations or products in coordination with the interface 32. Within the companion application, the interface 32 has a conversational agent interface to offer product recommendations. The system 1000 can have a combination of a hardware device with sensors to capturing user data and a companion mobile interface 32 on a separate hardware device to exchange data with the server 10. A hardware device with the companion mobile interface 32 can trigger a digital coaching session to recommend different products to complement styles and types of mental training activities (e.g. concentrative meditation, open-monitoring meditation, compassion meditation), physical activities (yoga, walking, spin etc.), peer coaching activities (e.g. discussions on various topics of emotional development, mirroring or eye gazing, practicing listening to a partner without speaking), and so on.

The server 10 can implement a state-based personality measure for the physical or emotional signature. State-based personality is a measurement that changes over a period of time based on collected data. Initially, server 10 can collect a brief trait personality measure. Then over time, through the collection of states, server 10 can dynamically re-compute the physical or emotional signature over the period of the time (e.g. at intervals, at detected events) of the user session so that it would be dynamically be changing based on the states over time during each user session. The server 10 can use a rolling average based on the states measured, for example.

The interface 32 can implement natural language generation techniques for communicating the product recommendations or output received from the server 10. The interface 32 can used advanced data points and user preferences, various types of psychographic and demographic data, transaction data on products linked to various healthy activities (running, yoga, etc.), and other contextual information on life goals and values. The interface 32 can use this data to further contextualize the output received from the server 10 to develop of tailored interface experience for the user.

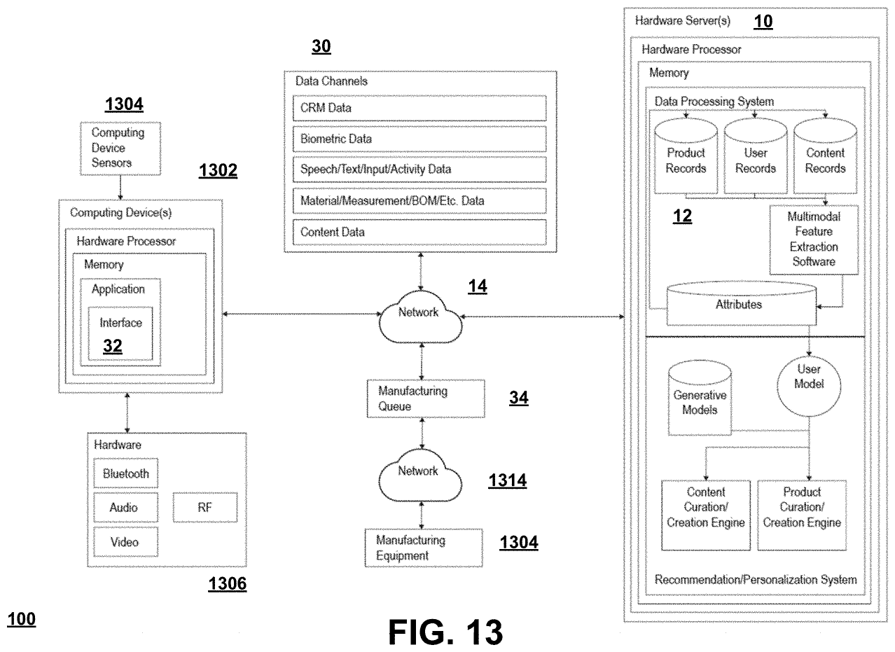

FIG. 13 illustrates an example system 100 that provides personalized products or product recommendations.

System 100 has one or more computing devices 1302 (with hardware processors 18), one of more hardware devices 1306, data channels 30, one or hardware servers 10, and manufacturing queue 34, which are communicatively coupled to one another via network 14. Manufacturing queue 34 is communicatively coupled to manufacturing equipment 1320 via another network 1314 such that the other elements of system 100 can only communicate with manufacturing equipment 1320 through manufacturing queue 34 and not directly.

The one or more computing devices 1302 have hardware processors 18 with computer readable non-transient memory storing an application providing interface 32. Users may interact with the interface, for example to provide user data or to request a product recommendation. Each computing device 1032 has one or more sensors 1304. These sensors may be used to collect user data, for example, physiological parameter data or cognitive-affective competency data. The sensor 1304 may be, for example, cameras, microphones, biometric sensors (heart monitor, blood pressure monitor, skin wetness monitor), location or position sensors, motion detection or motion capturing sensors,

The one more hardware devices 1306 can be used to collect user data. The hardware devices 1306 may be for example, a smart watch, a smart phone, a smart mirror, or any other smart exercise machine (e.g., connected stationary bike) equipped to collect user data. For example, a hardware device 1306 may have a camera and sensors to capture user data. The hardware devices 1306 are communicatively coupled to the computing devices 1302, for example via Bluetooth, RF, or other any other suitable communication protocol.

Data channels 30 can receive data from different data sources, such databases of products or different content providers (i.e., coaches, counsellors, influencers). Different channels 30 may provide different kinds of data. For example, there may be channels providing CRM data, biometric data, speech data, text data, input data, activity data, material data, measurement data, BOM data, and content data.

The one or more hardware servers 10 each have a hardware process with computer readable non-transient memory storing a data processing system (e.g. database 12 on non-transitory memory). The data processing system can receive data from the computing devices 1302, and data channels 30 and store this data in product records, user records, and content records. The data processing system has multimodal feature extracting software which is used to extract data from product records, user records and content records and store them in a database of attributes. The hardware servers 10 has a recommendation/personalization system having a user model, generative models, a content curation/creation engine and a production curation/creation engine. The attributes from database of attributes can be used as inputs for the user model which, along with the generative models are used to power the recommendation/personalization system's content curation/creation engine and product curation/creation engine.

In response to a request for a product or recommendation provided by a user at interface 32, the recommendation/personalization system, using the database of attributes, user model, and generative design models, curates or creates a product recommendation or personalized product. The product recommendation or personalized product will be displayed to the user at interface 32. If the product recommendation or personalized product is a product that can be manufactured, for example a garment, the user may request that the product be manufactured. The manufacturing request is sent to the manufacturing queue 34, along with any data from the data channels 30 or hardware servers 10 that are required to complete the request For example in the case of the garment, the manufacturing queue may require material data for the garment provided by the data channels 30, product data stored in the product records of the hardware servers 10, and the measurements and shipping information for the user stored in user records of the hardware servers 10. The manufacturing queue 34 provides instructions to the manufacturing equipment 1304 to manufacture the product.

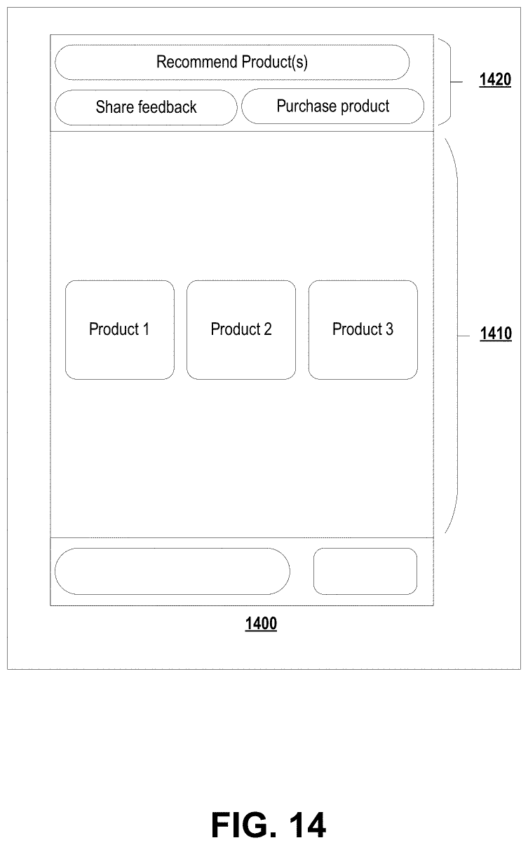

FIG. 14 illustrates an example of a user interface 1400 for product recommendations. The user interface 1400 also has selectable indicia 1420 that includes a selectable indicia to recommend one or more products to trigger a product request to update the user interface 1400 with visualizations of one or more product recommendations 1410. Upon selection of the selectable indicia 1420, the user interface 1400 can transmit a product request to the server 10, for example. In response, the user interface 1400 receives data for generating the visualizations of one or more product recommendations 1410 (which can include associated recommended content), and updates the user interface 1400 to display or communicate the product recommendations 1410 or associated recommended content. The product recommendations may include content that can be provided to the user as a message, image, or video in the user interface 1400. The messaging can also request additional input data (to be captured at user interface 1400) before generating the product recommendations 1410. The product recommendations 1410 are generated automatically by the sever 10, for example. The selectable indicia 1420 can include a selectable purchase option to select a recommended product for purchase. This can trigger instructions to be transmitted to a manufacturing queue 34 to manufacture the selected product of the product recommendations 1410. The selectable indicia 1420 can include a selectable feedback option to provide feedback data on a recommended product 1410 and/or a purchased product. The user interface 1400 can be stored on non-transitory computer readable medium and is executable by a hardware processor to implement operations described herein.

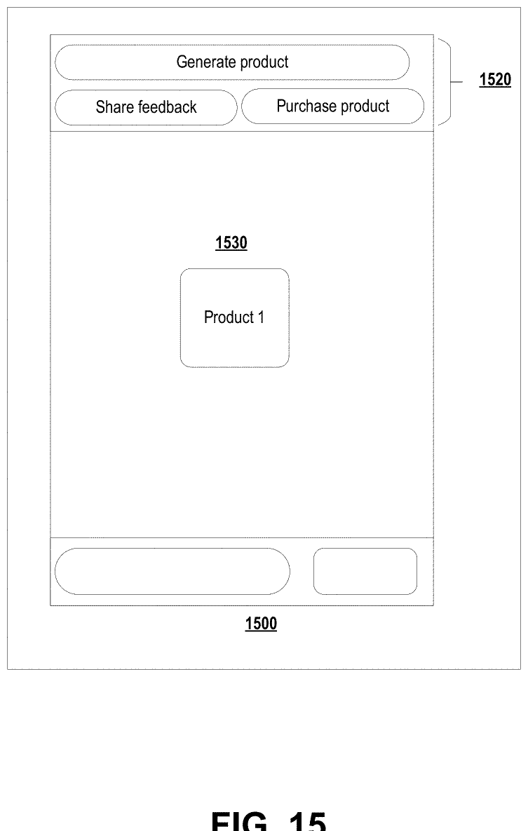

FIG. 15 illustrates an example of a user interface 1500 for product personalization. The user interface 1500 also has selectable indicia 1520 that includes a selectable indicia to generate one or more personalized products to trigger a product request to update the user interface 1500 with visualizations of one or more personalized products 1530. Upon selection of the selectable indicia 1520, the user interface 1500 can transmit a product request to the server 10, for example. In response, the user interface 1500 receives data for generating the visualizations of the one or more personalized products 1530 (which can include associated personalized content), and updates the user interface 1500 to display or communicate the one or more personalized products 1530 or associated recommended content. The product may include content that can be provided to the user as a message, image, or video in the user interface 1500. The messaging can also request additional input data (to be captured at user interface 1500) before generating the one or more personalized products 1530. The one or more personalized products 1530 are generated automatically by the sever 10, for example. The selectable indicia 1520 can include a selectable purchase option to select a recommended product for purchase. This can trigger instructions to be transmitted to a manufacturing queue 34 to manufacture the personalized product 1530. The selectable indicia 1520 can include a selectable feedback option to provide feedback data on the personalized product 1530. The user interface 1500 can be stored on non-transitory computer readable medium and is executable by a hardware processor to implement operations described herein.

In an aspect, there is provided a method for providing an interface for generating a product. The method involves storing an attributable database of product measurement records, movement features, perceptual preference features, physical and emotional signature features, user records, and generative design models in memory; capturing user data for a user session over a time period; in response to receiving a product request from an interface, select product category and product variables; extracting user attributes from the user data and associate with the product variables, the user attributes comprising at least one of measurement metrics, movement metrics, perceptual preference metrics, and physical and emotional signature metrics; computing target parameters for a target feel state for the user using the extracted user attributes; generating a product and associated manufacturing instructions by processing the extracted user attributes and the target parameters for the target feel state using the generative design models and the attributable database, wherein generating the product is based on an emotional signature and physical signature of the user; displaying a visualization of the product at the interface with a selectable purchase option; receiving purchase instructions for the product in response to selection of the selectable purchase option at the interface; transmitting manufacturing instructions for the product to a manufacturing queue to trigger production and delivery of the product; receiving feedback data on the product; and updating the attributable database or user model based on the feedback data.

In some embodiments, the method involves receiving a modification request for the product at the interface; and updating the product and the associated manufacturing instructions based on the modification request.

The word “a” or “an” when used in conjunction with the term “comprising” or “including” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

The terms “coupled”, “coupling” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through one or more intermediate elements or devices via an electrical element, electrical signal or a mechanical element depending on the particular context. The term “and/or” herein when used in association with a list of items means any one or more of the items comprising that list.

As used herein, a reference to “about” or “approximately” a number or to being “substantially” equal to a number means being within +/−10% of that number.

While the disclosure has been described in connection with specific embodiments, it is to be understood that the disclosure is not limited to these embodiments, and that alterations, modifications, and variations of these embodiments may be carried out by the skilled person without departing from the scope of the disclosure.

It is furthermore contemplated that any part of any aspect or embodiment discussed in this specification can be implemented or combined with any part of any other aspect or embodiment discussed in this specification.

1. A system for providing an interface for product personalization using a physical signature and emotional signature of a user, the system comprising:
non-transitory memory storing an attributable database of at least one of product measurement records, movement features, perceptual preference features, physical signature features, emotional signature features, user records, product records, and generative design models;
a hardware processor programmed with executable instructions for an interface to obtain user data for a user session over a time period, transmit a product request for the user session, display a visualization of a product generated for the user session in response to the product request, and receive quantitative and qualitative feedback data on the product;
a hardware server coupled to the memory to access the attributable database, the hardware server programmed with executable instructions to:
in response to receiving the product request from the interface, select product category and product variables;
extract user attributes from the user data for the user session and associate with the product variables, the user attributes comprising at least one of measurement metrics, movement metrics, perceptual preference metrics, physical signature metrics, emotional signature metrics, purchase history, and activity intent;
compute target parameters for a target feel state for the user using the extracted user attributes;
generate the product and associated manufacturing instructions by processing the extracted user attributes and the target parameters for the target feel state using the generative design models and the attributable database;
transmit the visualization of the product to the interface;
update the attributable database or user records based on the feedback data on the product;
a user device comprising one or more sensors for capturing the user data for the user session during the time period, and a transmitter for transmitting the captured user data to the interface of the hardware processor or the hardware server over the network to generate the product for the user session.

2. The system of claim 1 wherein the interface receives purchase instructions for the product, and wherein the hardware server transmits manufacturing instructions for the product in response to receiving purchase instructions.

3. The system of claim 1 wherein the hardware server generates the product and associated manufacturing instructions by generating bill of material files.

4. The system of claim 1 wherein the product comprises video content, and wherein the hardware server generates the product and associated code files by assembling content files for the video content.

5. The system of claim 1 wherein the interface receives a modification request for the product and wherein the hardware server updates the product and the associated manufacturing instructions based on the modification request.

6. The system of claim 1 wherein the user device captures the user data from a plurality of channels, wherein the user data comprises at least one of image data relating to the user, text input relating to the user, data defining physical or behavioural characteristics of the user, and audio data relating to the user, wherein the hardware server extracts product variables or attributes using multimodal feature extraction and classifies the product variables and attributes, and wherein the hardware server classifies different types of data streams for the user data for multimodal feature extraction.

7. The system of claim 6 wherein the hardware server is programmed with executable instructions to compute activity metrics, cognitive-affective competency metrics, and social metrics using the user data for the user session and the user attributes by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis; compute one or more states of one or more cognitive-affective competencies of the user based on the cognitive-affective competency metrics and the social metrics; compute the emotional signature metrics of the user based on the one or more states of the one or more cognitive-affective competencies of the user; and generating the product based on at least one of the emotional signature of the user, activity metrics, product records, and/or the user records.

8. The system of claim 1 wherein the hardware server generates the measurement metrics using at least one of 3D scanning, machine learning prediction, and user measuring, wherein the product comprises a garment, and wherein the hardware server generates the measurement metrics using garment measuring to capture garment data for the product data.

9. The system of claim 1 wherein the hardware server generates the movement metrics based on at least one of inertial measurement unit (IMU) data, computer vision, pressure data, radio-frequency data.

10. The system of claim 1 wherein the hardware server extracts the user attributes from user data comprising at least one of purchase history, activity intent, interaction history, and review data for the user.

11. The system of claim 1 wherein the hardware server generates the perceptual preference metrics based on at least one of garment sensation, preferred hand feel, thermal preference, and movement sensation.

12. The system of claim 1 wherein the hardware server generates the emotional signature metrics based on at least one of personality data, mood state data, emotional fitness data, personal values data, goals data, and physiological data.

13. The system of claim 1 wherein the hardware processor computes a preferred sensory state as part of the extracted user attributes, receives object identification data and computes a preferred sensory state as part of the object identification data.

14. The system of claim 1 wherein the hardware server computes social signature metrics, connectedness metrics, and/or resonance signature metrics.

15. The system of claim 1 wherein the user device connects to or integrates with an immersive hardware device that captures audio data, the image data and data defining physical or behavioural characteristics of the user as part of the user data.

16. The system of claim 1 wherein the non-transitory memory has a content repository and the hardware server has a content curation engine that generates content as part of the product and transmits the generated content to the interface, wherein the product comprises content for display or playback on the hardware processor or the user device.

17. The system of claim 1 wherein the product relates to a garment, wherein the attributable database comprises simulated garment records, wherein the hardware server generates simulated product options as part of the product and associated manufacturing instructions, and wherein the interface displays a visualization of the simulated product options, wherein the simulated product options comprise at least one of soft body physics simulation, hard body physics simulation, static 3D viewer and AR/VR experience content.

18. A method for providing an interface for generating a product, the method comprising:
storing an attributable database of product measurement records, movement features, perceptual preference features, physical and emotional signature features, user records, and generative design models in memory;
capturing user data for a user session over a time period;
in response to receiving a product request from an interface, select product category and product variables;
extracting user attributes from the user data and associate with the product variables, the user attributes comprising at least one of measurement metrics, movement metrics, perceptual preference metrics, and physical and emotional signature metrics;
computing target parameters for a target feel state for the user using the extracted user attributes;
generating or recommending a product by processing the extracted user attributes and the target parameters for the target feel state using the attributable database, wherein generating or recommending the product is based on an emotional signature and physical signature of the user;
displaying a visualization of the product at the interface with a selectable purchase option;
in response to selection of the selectable purchase option at the interface; transmitting instructions for the product to trigger production or delivery of the product;
receiving feedback data on the product; and
updating the attributable database or user model based on the feedback data.

19. A system for providing an interface with product recommendations, the system comprising:
non-transitory memory storing an attributable database of at least one of product measurement records, movement features, perceptual preference features, physical and emotional signature features, user records, and generative design models;
a hardware processor programmed with executable instructions for an interface to obtain user data for a user session over a time period, transmit a product request for the user session, provide product recommendations for the user session in response to the product request, receive a selected product of the product recommendations; and receive feedback data on the selected product;
a hardware server coupled to the memory to access the attributable database, the hardware server programmed with executable instructions to:
in response to receiving the product request from the interface, select product category and product variables;
extract user attributes from the user data for the user session and associate with the product variables, the user attributes comprising at least one of measurement metrics, movement metrics, perceptual preference metrics, and physical and emotional signature metrics;
compute target parameters for a target feel state for the user using the extracted user attributes;
compute product recommendations using a recommendation system to process the extracted user attributes and the target parameters for the target feel state, the product recommendations computed using an emotional signature and a physical signature;
transmit the product recommendations to the interface over a network;
receive notification of the selected product from the interface;
receive feedback data on the selected product; and
update the attributable database based on the feedback data on the selected product;
at least one data channels with one or more sensors for capturing user data during the time period, and a transmitter for transmitting the captured user data to the interface of the hardware processor or the hardware server over the network to compute the product recommendations.

20. The system of claim 19, wherein the user attributes comprising quantitative user attributes of physical metrics and qualitative user attributes, the qualitative user attributes comprising the emotional signature metrics and/or perceptual preferences of the user.
